(19) United States
(12) Patent Application Publication
(10) Pub. No.: US 2016/0010155 A1
(43) Pub. Date: Jan. 14, 2016

(54) KCNK3 Channel Loss-of-Function Mutants in Familial and Idiopathic Pulmonary Arterial Hypertension

(71) Applicant: THE TRUSTEES OF COLUMBIA UNIVERSITY IN THE CITY OF NEW YORK, New York, NY (US)

(72) Inventors: Robert Kass, New York, NY (US); Kevin SAMPSON, New York, NY (US); Danilo Roman CAMPOS, Belo Horizonte (BR); Lijiang MA, Rego Park, NY (US); Wendy Kay CHUNG, Hackensack, NJ (US)

(21) Appl. No.: 14/769,440

(22) PCT Filed: Feb. 21, 2014
(86) PCT No.: PCT/US14/17851

§ 371 Date: Aug. 20, 2015

Related U.S. Application Data
(60) Provisional application No. 61/767,768, filed on Feb. 21, 2013.

(51) Int. Cl. C12Q 1/68; A61K 45/06; A61K 31/08; A61K 31/02; C07K 14/705; A61K 31/192

(52) U.S. Cl. CPC C12Q 1/6883; C07K 14/705; A61K 31/192; A61K 31/08; A61K 31/02; A61K 45/06; C12Q 2600/156; C12Q 2600/112

(57) ABSTRACT

Pulmonary arterial hypertension (PAH) is a devastatig disease with high mortality. Familial cases of PAH are usually characterized by autosomal dominant transmission with reduced penetrance, and mutations in Bone Morphogenetic Protein receptor type 2 (BMPR2), account for approximately 70% of familial cases, but some familial cases are of unknown genetic etiology. A novel heterozygous missense variant c.608 G>A, G203D in the KCNK3 (potassium channel subfamily K, member 3) gene was identified as a disease causing candidate gene in the family. Five additional heterozygous missense variants were independently identified in 92 unrelated familial PAH and 230 idiopathic PAH patients, genetically independently confirming the results in the first family. All six novel variants were located in highly conserved domains and were predicted to be damaging. Electrophysiological studies of the channel indicated that these missense mutations all result in loss of function, and some mutations were rescued by the phospholipase inhibitor ONO RS-082.

### CROSS-REFERENCE TO RELATED APPLICATIONS

This application claims benefit of Provisional Application Ser. No. 61/767,768, filed Feb. 21, 2013, the entire contents of which are hereby incorporated by reference as if fully set forth herein, under 35 U.S.C. §119(e).

### STATEMENT OF GOVERNMENTAL INTEREST

This invention was made with government support under Grant No. DK57539 awarded by the National Institutes of Health. The government has certain rights in the invention.

### BACKGROUND OF THE INVENTION

Pulmonary arterial hypertension (PAH) is a rare devastating disease with high mortality. Familial cases of PAH are usually characterized by autosomal dominant transmission with reduced penetrance, and mutations in Bone Morphogenetic Protein Receptor type 2 (BMPR2), account for approximately 70% of familial cases, but some familial cases are of unknown genetic etiology. PAH is characterized by increased pulmonary artery pressure in the absence of common causes of pulmonary hypertension such as heart, lung and thrombo-embolic chronic diseases.¹ Patients with idiopathic and familial PAH have historically had an estimated median survival of 2.8 years, with 1-year, 3-year, and 5-year survival rates of 68%, 48%, and 34%, respectively,² prior to availability of novel therapies. In the modern management era, PAH remains a progressive, fatal disease. Clinical presentations can be nonspecific, and patients are often diagnosed late in their clinical course.

The etiology of PAH is heterogeneous, and some cases are familial. Molecular genetic studies have demonstrated that BMPR2 mutations are present in ˜70% of cases of familial PAH, as well as 10-25% of cases of idiopathic PAH.³⁻⁵ PAH may also occur in patients carrying ALK1 (and more rarely ENG) mutations, which are known causes of hereditary hemorrhagic telangiectasia.³′⁶⁻⁹ Rarely, mutations in SMAD9 have been identified in idiopathic PAH.^{10,11} Novel mutations in Caveolin-1 (CAV1) in patients with familial and idiopathic PAH have been previously identified.¹² Approximately 25% of patients with familial PAH do not have an identifiable genetic etiology.

Therefore, a need exists for the development of effective therapies and prediction of disease and therapy for PAH for patients that do not express the BMPR2 mutations.

### SUMMARY OF THE INVENTION

Certain embodiments are directed to a cDNA encoding one or more loss-of-function variants of a gene encoding KCNK3 having NM 002246 (SEQ. ID. NO. 1) selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

Other embodiments are directed to a method comprising a) obtaining a biological sample from a subject, b) determining if the biological sample (i) comprises a variant of the human KCNK3 channel gene having Gene Accession Number NM 002246, or (ii) has significantly reduced KCNK3 function compared to a biological sample from a normal subject, or (iii) expresses an mRNA encoding an abnormal form of KCNK3 protein, or (iv) expresses a functionally abnormal KCNK3 channel protein, c) determining that the subject is at risk for developing PAH if (i), (ii), (iii) or (iv) are satisfied in step b)., and d) if it is determined that the subject is at risk for developing PAH, then monitoring the subject for signs of PAH or treating the subject for PAH or both. The subject can be either asymptomatic or symptomatic, for example a symptomatic subject may have pulmonary arterial pressure. In certain embodiments the subject has a familial history of PAH.

In embodiments of the methods, treatment for PAH comprises prophylactic administration of a therapeutically effective amount of a KCNK3 channel agonist, including a KCNK3 channel agonist that is a member selected from the group consisting of ONO-RS-82, Treprostinil, Isoflurane and Halothane. In preferred embodiments of the invention, the subject is human. The subject may have a personal or family history of heart disease.

Other embodiments include methods comprising a) identifying a subject as having one or more symptoms of PAH, b) obtaining a biological sample from the subject, c) determining if the biological sample (i) comprises a loss-of-function variant of the Human KCNK3 channel gene having Gene Accession Number NM 002246, or (ii) has significantly reduced KCNK3 function compared to a biological sample from a normal subject, or (iii) expresses an mRNA encoding an abnormal form of KCNK3 protein, or (iv) expresses a functionally abnormal KCNK3 channel protein, d) if it is determined that the biological sample from the subject satisfies (i), (ii), (iii),or (iv), and the subject has increased pulmonary arterial pressure, then 0 diagnosing the subject as having PAH and treating the subject for PAH. In some embodiments the abnormal form of KCNK3 protein is a protein that has a characteristic selected from the group consisting of reduced potassium channel current at physiological pH, abnormal function or significantly reduced K+ channel current, abnormal topology based on the crystal structure of TWIK-1 channel, or reduced K⁺ selectivity.

In yet other embodiments methods are included comprising a) identifying a subject diagnosed with a disease or condition associated with significantly reduced KCNK3 function compared to a normal subject, and b) administering to the subject a therapeutically effective amount of a KCNK3 channel agonist thereby treating the subject for the disease or condition.

Other embodiments include cells transfected with a cDNA encoding a loss-of-function variant selected from the group consisting of G203D, G97R, V221L, T8K, E182K, and Y192C.

In another embodiment a method comprises a) identifying a subject having one or more symptoms of PAH, b) obtaining a biological sample from the subject, c) determining if the biological sample has significantly reduced KCNK3 function compared to a biological sample from a normal subject, d) if it is determined that the biological sample from the subject satisfies c), then diagnosing the subject as at risk for developing PAH and monitoring the subject for symptoms of PAH or treating the subject for PAH for example by administering the subject a therapeutically effective amount of a KCNK3 channel agonist that increases or normalizes KCNK3 function and increases pulmonary vasodilation. In certain embodiments, the therapeutically effective amount of a KCNK3 agonist ranges from about 0.0001 to 100 mg/kg/day.

Other embodiments are directed to a pharmaceutical formulation and kits comprising them comprising two or more KCNK3 channel agonists, in therapeutically effective amounts that increase KCNK3 channel current in a cell (e.g., pulmonary artery smooth muscle cell).

Other embodiments are directed to microarrays comprising one or more oligonucleotide probes bound to a support which probes are complementary to and hybridize to one or more respective target oligonucleotide capable of selectively hybridizing to one or more nucleic acid molecules having a single nucleotide polymorphism (SNP) selected from the group consisting of selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

Other embodiments are directed to SNP detection kits, including but not limited to, packaged probe and primer sets (e.g., TaqMan probe and primer sets), arrays and/or microarrays of nucleic acid molecules, and beads that contain one or more probes, primers, or other detection reagents for detecting one or more SNPs of the present invention. The kits can optionally include various electronic hardware components, containers, and devices.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a diagram that illustrates an example pedigree of familial PAH. Segregation of KCNK3 mutation c.608 G>A G203D in FPPH050 is indicated, according to an embodiment;

FIG. 2 is a diagram that illustrates another example pedigree of familial PAH. Segregation of KCNK3 mutation c.289G>A G97R in PSL3555 is indicated, according to an embodiment;

FIG. 3 is a diagram that illustrates another example pedigree of familial PAH. Segregation of KCNK3 mutation c.661G>C V221L in TNN001 is indicated, according to an embodiment;

FIG. 4 is an example micrograph that illustrates the histopathology of explanted lungs from idiopathic PAH patient JM0041, according to an embodiment;

FIG. 5 is an example micrograph that illustrates the histopathology of explanted lungs from idiopathic PAH patient JM0041, according to another embodiment;

FIG. 6 is an example micrograph that illustrates the histopathology of explanted lungs from idiopathic PAH patient JM0041, according to another embodiment;

FIG. 7 is an example micrograph that illustrates the histopathology of explanted lungs from idiopathic PAH patient JM0041, according to another embodiment;

FIG. 8A is a diagram that shows the topology of hKCNK3 channel and the position of mutations T8K, G97R, E182K, Y192C, G203D and V221L, according to an embodiment;

FIG. 8B is a diagram that illustrates the alignment of KCNK3 channel family and TWIK-1 channel according to an embodiment;

FIG. 8C is a graph that illustrates example wild type (WT) pH dependence of TWIK-1 channel current, according to an embodiment;

FIG. 8D are graphs that illustrates example current traces at pH 7.4 for WT hKCNK3 and T8K, G97R, E182K, Y192C, G203D and V221 mutants hKCNK3 channel, according to an embodiment;

FIG. 8E is a bar graph that illustrates the example results illustrated in FIG. 8D, according to an embodiment;

FIG. 8F is a bar graph that illustrates an example comparison between homozygous and heterozygous expression of WT hKCNK3 with Y192C, G203D and V221L mutants, according to an embodiment;

FIG. 9A is a graph that illustrates example pharmacology recovery of mutant KCNK3 channels where representative recordings show before and after ONO RS-082 application, according to an embodiment;

FIG. 9B is a graph that illustrates an example time-course of drug application before, during, and after drug application according to an embodiment;

FIG. 9C is a graph that illustrates an example summary of results of drug effects on the KCNK3 channel before and maximal drug response respectively, according to an embodiment; and

FIG. 10 is a flow chart that illustrates an example method for diagnosing and treating PAH, according to an embodiment.

### DETAILED DESCRIPTION

Six novel loss-of-function variants of the KCNK3 ion channel gene have been discovered that are associated with an increased risk for developing PAH. The presence of one or more of these loss-of function variants can be used in new methods for diagnosing asymptomatic and symptomatic subjects for PAH, and then monitoring and/or treating the if it is determined that the subject is at risk for developing PAH or a diagnosis of PAH is confirmed. Rather than treat PAH conventionally, it is now possible to treat such subjects with the administration of a KCNK3 agonist. Certain embodiments of the invention are directed to cDNAs encoding the six novel loss-of-function variants of the KCNK3 ion channel gene, to microarrays for identifying the six novel loss-of-function variants in a biological sample, and to methods for diagnosing and treating PAH.

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

A family with multiple affected individuals with PAH without identifiable mutations in BMPR2, ALK1, ENG, SMAD9, or CAV1 was studied with whole exome sequencing (WES). Additional familial and idiopathic PAH patients were screened for the mutations in the gene identified by WES. All variants were expressed in COS-7 cells and channel function was studied by patch clamp.

WES identified six different loss-of-function mutations in the KCNK3 channel (potassium channel subfamily K, member 3, also referred to as the “TASK-1 channel”) in subjects with either familial or idiopathic PAH. The human KCNK3 gene sequence is identified in Gene Accession Number NM 002246. Six heterozygous missense variants of the Human KCNK3 channel gene were independently identified from studies of 92 unrelated familial PAH and 230 idiopathic PAH patients, which genetically independently confirmed the results in the first family. All six novel variants were located in highly conserved domains of the channel and electrophysiological studies in COS-7 cells, indicated that these missense mutations all resulted in loss-of-function in the KCNK3 channel.

Certain of these newly-identified loss-of-function mutations were rescued by the KCNK3 agonist, phospholipase inhibitor ONO RS-082, indicating that this drug and other KCNK3 agonists can be used therapeutically to treat PAH and other diseases associated with loss-of-function KCNK3 mutations.

Some example embodiments of the invention are described below in the context of identifying the six KCNK3 variants or to cDNA that incorporates at least one of the variants. However, the invention is not limited to this context. In other embodiments it is possible to determine if an asymptomatic subject has PAH, or if a subject is at risk for developing PAH, identifying a subject diagnosed as having one or more symptoms of PAH, or identifying a subject diagnosed with PAH. In other embodiments, pharmaceutical formulations of two or more KCNK3 channel agonists can be administered in therapeutically effective amounts to treat PAH or other disease associated with a significant loss of KCNK3 function. In various embodiments, such determinations include determining if the biological sample (i) comprises a loss-of-function variant of the human KCNK3 channel gene having Gene Accession Number NM 002246, or (ii) has significantly reduced KCNK3 function compared to a biological sample from a normal subject, or (iii) expresses an mRNA encoding an abnormal form of KCNK3 protein, or (iv) expresses a functionally abnormal KCNK3 channel protein.

### 1. DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N. Y. (2000). Unless defined otherwise, all technical and scientific terms used herein have the same meaning as commonly understood by one of ordinary skill in the art.

The terms used herein have the meanings in the following table.

| | |
|---|---|
| Administering | May be performed using any of the various methods of delivery |
| | systems well known to those skilled in the art. The administering can |
| | be performed, for example, orally, parenterally, intraperitoneally, |
| | intravenously, intraarterially, transdermally, sublingually, |
| | intramuscularly, rectally, transbuccally, intranasally, liposomally, via |
| | inhalation, vaginally, introccularly, via local delivery, subcutaneously, |
| | intraadisposally, intraarticularly, intrathecally, into a cerebral |
| | ventricle, intraventricularly, intratumorally, into cerebral parenchyma |
| | or intraparenchymally or microinjection. |
| Biological sample | Any biological specimen obtained from a subject. Samples include, |
| | without limitation, a tissue sample (e.g., tumor tissue) such as a biopsy |
| | of a tumor, and cellular extracts thereof, whole blood, plasma, serum, |
| | red blood cells, white blood cells (e.g., peripheral blood mononuclear |
| | cells), saliva, urine, stool (i.e., feces), tears, nipple aspirate, lymph, |
| | fine needle aspirate, any other bodily fluid. In some embodiments, the |
| | sample is whole blood or a fractional component thereof such as |
| | plasma, serum, or a cell pellet. |
| KCNK3 channel | Potassium channel subfamily K, member 3, also referred to as the |
| | TASK-1 channel is a protein that in humans is encoded by the KCNK3 |
| | gene. This gene encodes K_{2P}3.1, one of the members of the |
| | superfamily of potassium channel proteins containing two pore- |
| | forming P domains. K_{2P}3.1 is an outwardly rectifying channel that is |
| | sensitive to changes in extracellular pH and is inhibited by |
| | extracellular acidification. Also referred to as an acid-sensitive |
| | potassium channel, it is activated by the anesthetics halothane and |
| | isoflurane. Although three transcripts are detected in northern blots, |
| | there is currently no sequence available to confirm transcript variants |
| | for this gene. |
| | The KCNK3 (TASK-1) channel encodes a pH-sensitive potassium |
| | channel in the two pore domain superfamily that is characterized by 4 |
| | transmembrane domains and 2 pore domains per subunit.²⁰ KCNK3 |
| | channels lack voltage dependence and are often thought of as ‘leak |
| | channels.’¹⁸ A decrease in leak K1 conductance in the KCNK3 channel |
| | leads to cell depolarization that enhances the probability that the L- |
| | type Ca channels in smooth muscle cells are open, causing periodic |
| | Ca21 entry and vasoconstriction, a factor that contributes to PAH. The |
| | function of this channel is to conduct K+ current, maintain resting |
| | membrane potential and to regulate vascular tone of pulmonary artery |
| | smooth muscle cells; and thus KCNK3 channels contribute to arterial |
| | smooth muscle cell relaxation.^{14-18,21} |
| KCNK3 channel | A KCNK3 channel variant is used herein synonymously with KCNK3 |
| variant | channel mutation. |
| Subject | A mammal, typically a human, but optionally a mammalian animal of |
| | veterinary importance, including but not limited to horses, cattle, |
| | sheep, dogs, and cats. |
| Significant Loss of | A reduction of K+ current through the KCNK3 channel of at least |
| KCNK3 function | 10% compared to normal current levels. |
| Therapeutically | An amount that achieves the intended therapeutic effect of reducing or |
| effective amount | eliminating PAH in a subject thereby treating it. The full therapeutic |
| | Effect does not necessarily occur by administration of one dose and |
| | may occur only after administration of a series of doses. Thus, a |
| | therapeutically effective amount may be administered in one or more |
| | administrations. |
| Genetic | Susceptibility of a subject to a disease, such as PAH. Detecting a |
| predisposition | genetic predisposition includes detecting the risk of developing the |
| | disease, and determining the susceptibility of that subject to |
| | developing the disease or to having a poor prognosis for the disease. |
| | Thus, if a subject has a genetic predisposition to a disease they do not |
| | necessarily develop the disease but are at a higher than normal risk for |
| | developing the disease. |
| Gene | A segment of DNA that contains the coding sequence for a protein, |
| | wherein the segment may include promoters, exons, introns, and other |
| | untranslated regions that control expression. |
| Genotype | A genotype is the genetic makeup of a cell, an organism, or an |
| | individual (i.e. the specific allele makeup of the individual) usually |
| | with reference to a specific character under consideration. |
| Locus | A location on a chromosome or DNA molecule corresponding to a |
| | gene or a physical or phenotypic feature, where physical features |
| | include polymorphic sites. |
| Mutation | Any change of a nucleic acid sequence as a source of genetic |
| | variation. |
| Odds Ratio | A calculation performed by analysis of a two by two contingency |
| | table. In one example, the first column provides a risk indicator in the |
| | absence of a disease (e.g., IgAN). The second column provides the |
| | same risk indicator in the presence of the same disease. The first row |
| | lists the risk indicator in the absence of a risk factor (such as race) and |
| | the second row lists the same risk indicator in the presence of the same |
| | risk factor (i.e., race). The Odds Ratio (OR) is determined as the |
| | product of the two diagonal entries in the contingency table divided by |
| | the product of the two off-diagonal entries of the contingency table. |
| | An OR of 1 is indicative of no association. Accordingly, very large or |
| | very small ORs are indicative of a strong association between the |
| | factors under investigation. The OR is independent of the ratio of |
| | cases or controls in a study, group or subset. |
| Polymorphism | A variation in a gene sequence. Polymorphisms can be referred to, for |
| | instance, by the nucleotide position at which the variation exists, by |
| | the change in amino acid sequence caused by the nucleotide variation, |
| | or by a change in some other characteristic of the nucleic acid |
| | molecule or protein that is linked to the variation. In the instant |
| | application “polymorphism” refers a traditional definition meaning |
| | that the minor allele frequency must be greater than at least 1%. |
| SNP or SNV | A “single nucleotide polymorphism (SNP)” or a “single nucleotide |
| | variant (SNV)” as used herein both mean a single base (nucleotide) |
| | polymorphism in a DNA sequence among individuals in a population. |
| | SNP/SNV genotyping is the measurement of genetic variations of |
| | SNPs/SNVs between members of a species. SNPs are one of the most |
| | rish all common types of genetic variation. An SNP is a single base |
| | pair mutation at a specific locus, usually consisting of two alleles |
| | (where the rare allele frequency is >1%). |

### 2. OVERVIEW

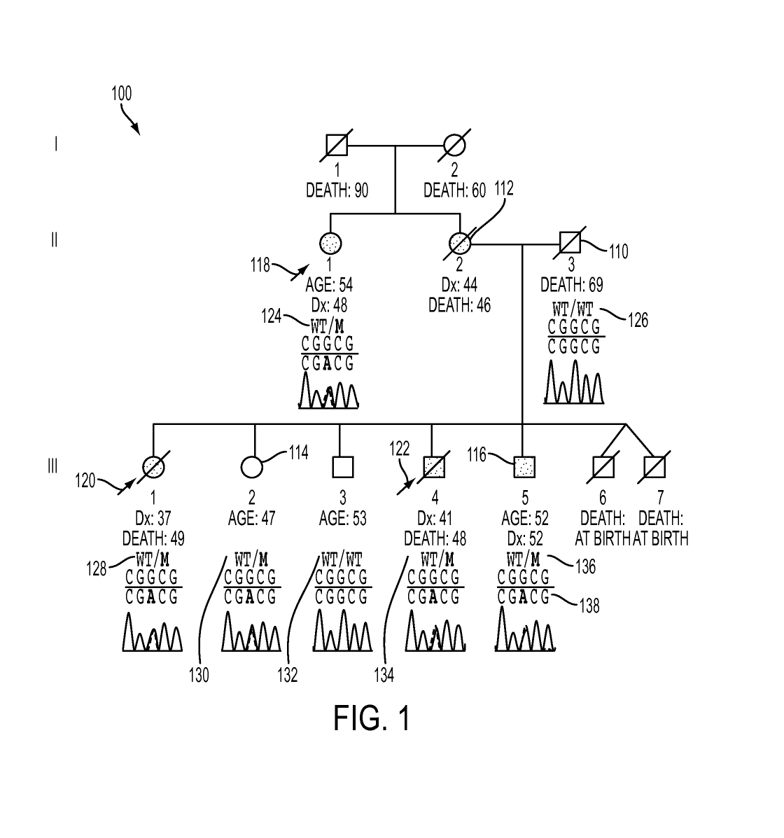

FIG. 1 is a diagram 100 that illustrates an example pedigree of familial PAH. Segregation of KCNK3 mutation c.608 G>A G203D in FPPH050 is indicated, according to an embodiment. The sequence variants were filtered for those that were rare (<1% frequency), shared by affected family members as heterozygous mutations given the autosomal dominant mode of transmission in the family, and prioritizing the rare variants based upon predicted pathogenic effects on the protein using 7 different prediction algorithms. Segregation analysis was performed on all available family members. FIG. 1 represents confirmation of a gene mutation in the family where II.3 in the pedigree 110 did not carry the c.608 G>A, G203D in KCNK3 novel variant, indicating that the affected II.2 in the pedigree 112 is an obligate carrier. II.2 in the pedigree 114 and III.5 in the pedigree 116 carry the c.608 G>A, G203D in KCNK3. Although III.5 in the pedigree 116 was initially unaffected when initially recruited, he has subsequently developed PAH. III.2 in the pedigree 114 remains unaffected. One hundred ethnically matched unrelated unaffected control caucasian DNA samples were genotyped, and the c.608 G>A KCNK3 variant was not present in any of the 100 controls. The c.608 G>A, 138 G203D KCNK3 variant is predicted to be damaging by SIFT and POLYPHEN2 and is located in the highly conserved second potassium channel pore region. Arrows 118, 120, and 122 show the subjects analyzed using whole exome sequencing. Genotypes of family members are shown under every symbol with “WT/WT” 126 and 132 and denote wild type and “WT/M” 124, 128, 130, 134 and 136 denotes one copy of the KCNK3 mutation.

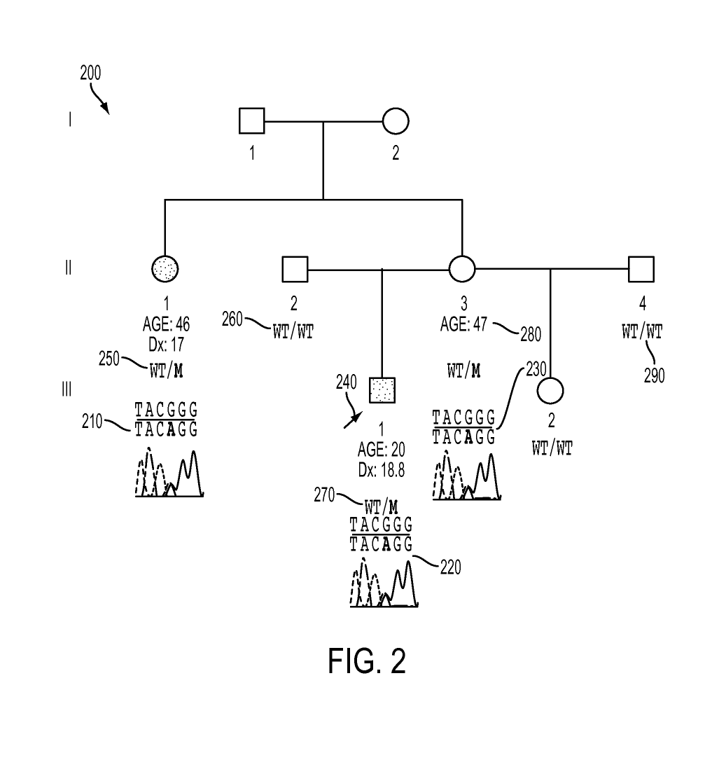

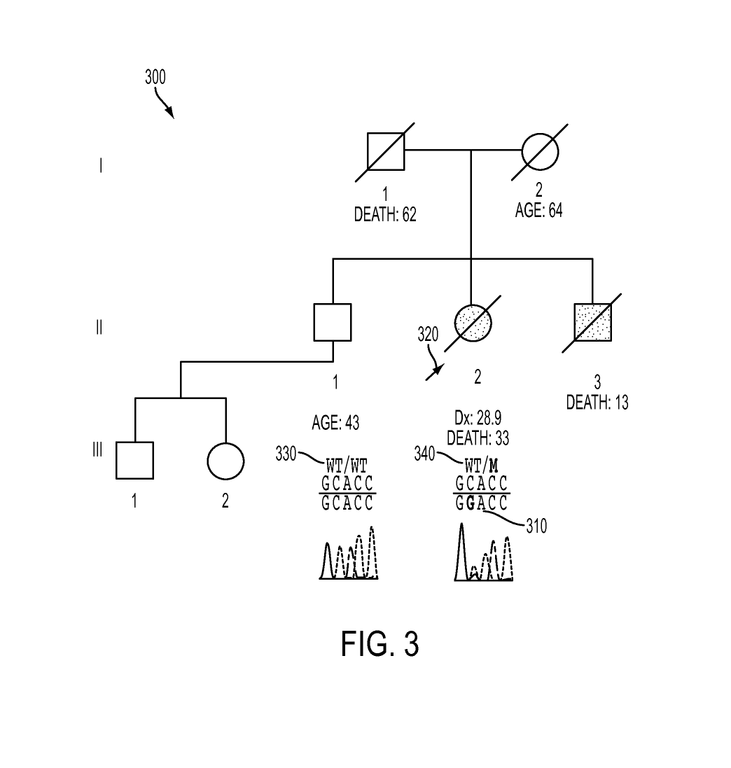

FIG. 2 and FIG. 3 are diagrams 200 and 300 respectively both of which illustrate ten additional probands with familial PAH studied by WES. FIG. 2 200 is a diagram that illustrates another example pedigree of familial PAH. Segregation of KCNK3 mutation c.289 G>A G97R in PSL3555 is indicated, according to an embodiment. FIG. 3 300 is a diagram that illustrates another example pedigree of familial PAH. Segregation of KCNK3 mutation c.661 G>C V221L in TNN011 is indicated, according to an embodiment. Two heterozygous novel KCNK3 variants G97R and V221L were identified in two of these families. These variants were confirmed by Sanger sequencing and tested in available family members and segregated with disease. An additional 82 unrelated probands with familial PAH and 230 patients with idiopathic PAH were screened for mutations in KCNK3. Three novel heterozygous amino acid substitutions T8K, E182K and Y192C were identified among the 230 idiopathic PAH patients. The six variants were all predicted to be damaging. The frequency of KCNK3 mutations in the idiopathic PAH cases was 1.30% (3/230) and 3.2% in familial cases (3/93). Segregation of KCNK3 mutations c.289G>A G97R in PSL3555 210, 220, and 230 are indicated in FIG. 2. Segregation of KCNK3 mutations c.661G>C V221L in TNN001 310 are indicated in FIG. 3. Arrows 240 in FIG. 2 and arrows 320 in FIG. 3 show the subjects analyzed using whole exome sequencing. Genotypes of family members are shown under every symbol with “WT/WT” 260 and 290 in FIGS. 2 and 330 in FIG. 3 and denote wild type and “WT/M” 250, 270, and 280 in FIGS. 2 and 340 in FIG. 3 and denotes one copy of the KCNK3 mutation.

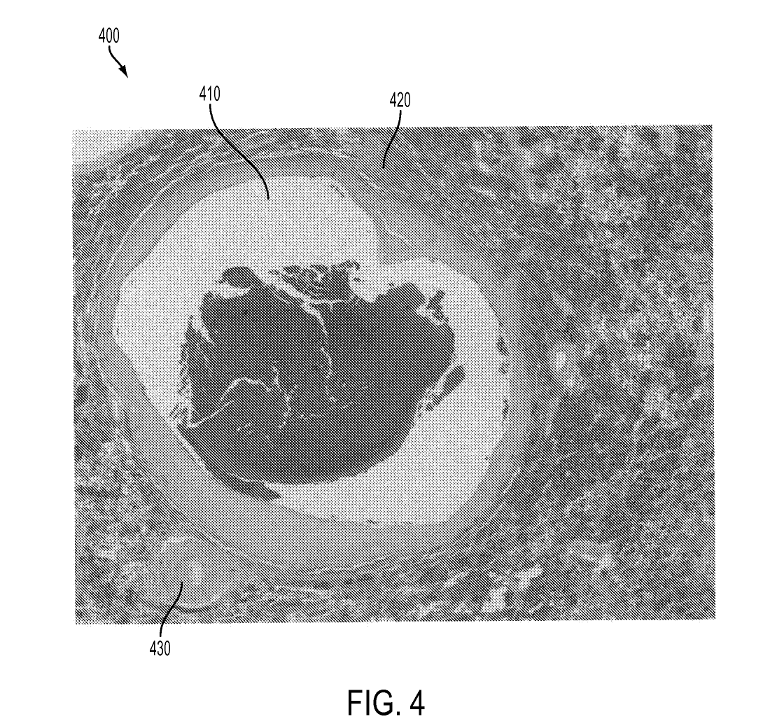

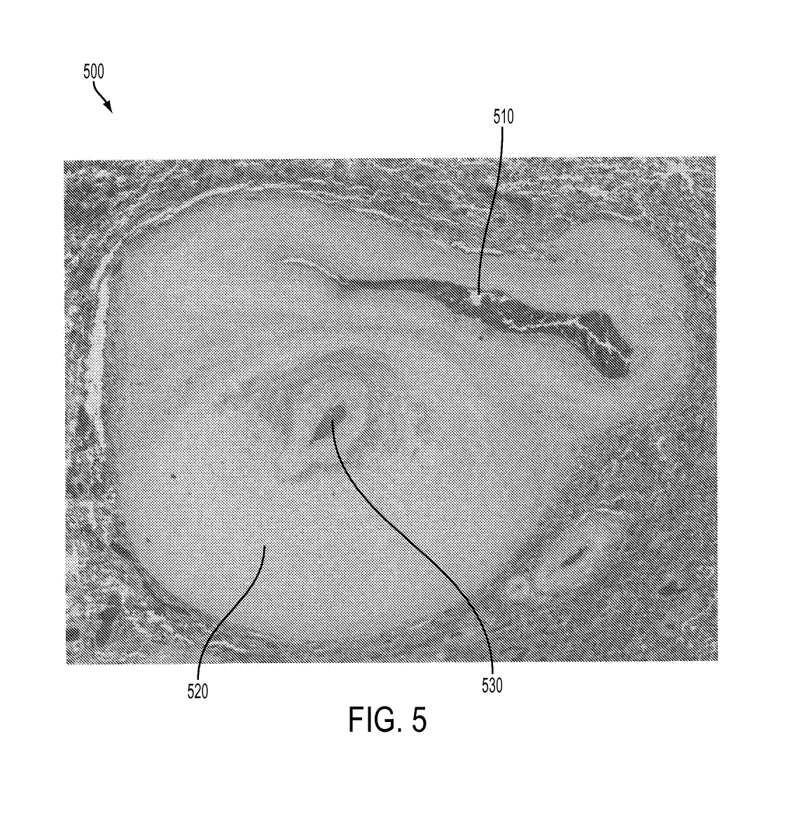

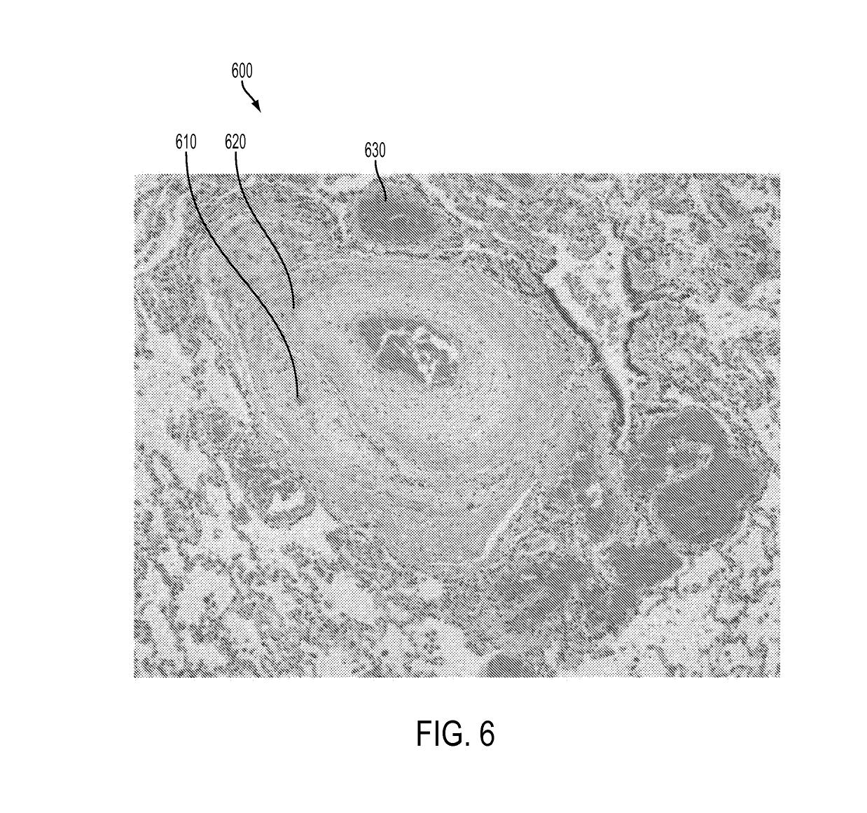

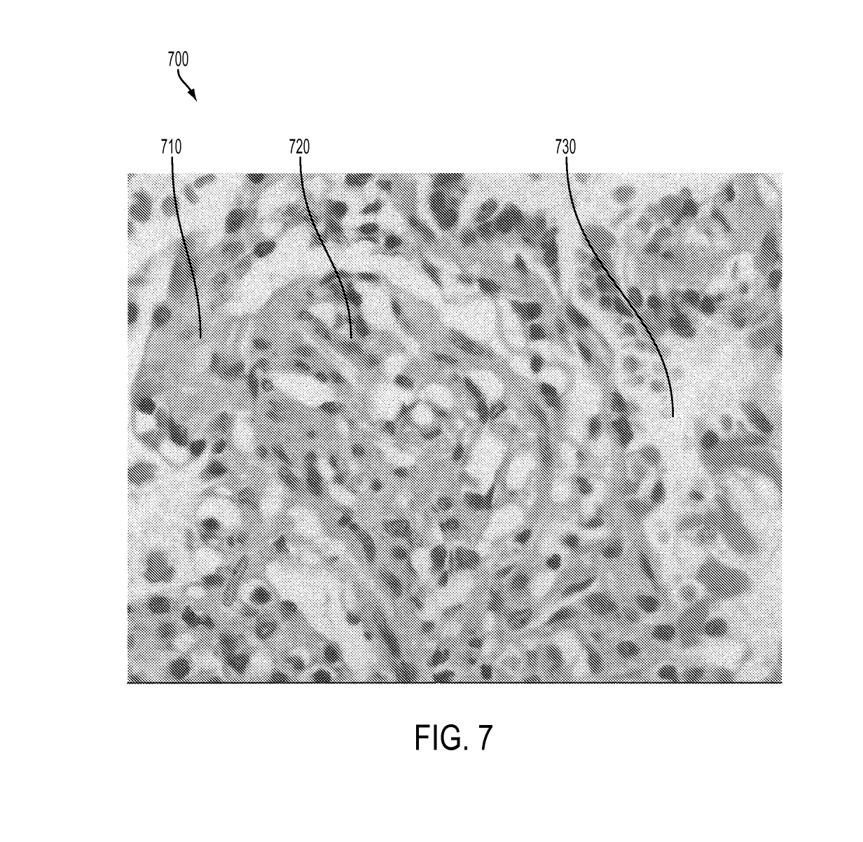

FIG. 4, FIG. 5, FIG. 6, and FIG. 7 illustrate histopathology from two patients with lung transplants FIG. 4 400 is an example micrograph that illustrates the histopathology of explanted lungs from idiopathic PAH patient JM0041 with a concentrically dilated arterial vessel compressing the adjacent airway and intimal proliferation. Histopathology of explanted lungs from idiopathic PAH patient JM0041 illustrates a concentrically dilated arterial vessel 410 that compresses the adjacent airway 420 and intimal proliferation 430. FIG. 5 500 is an example micrograph that illustrates the histopathology of explanted lungs from idiopathic PAH patient JM0041 with eccentric aneurysmal dilatation of an artery 510 that contains extensive fibrointimal proliferation illustrated 520 and shows marked reduction of luminal diameter 530. FIG. 6 600 is an example micrograph that illustrates the histopathology of explanted lungs from idiopathic PAH patient JM0041 with pulmonary artery branch recanalization 610, fibrointimal proliferation 610 and adjacent vascular dilatation 620 of an angiomatoid type lesion of PAH are illustrated in. FIG. 7 700 is an example micrograph that illustrates the histopathology of explanted lungs from idiopathic PAH patient JM0041 with a small pulmonary artery 710 having endothelial 720 and intimal 730 proliferation of a plexiform arteriopathy.

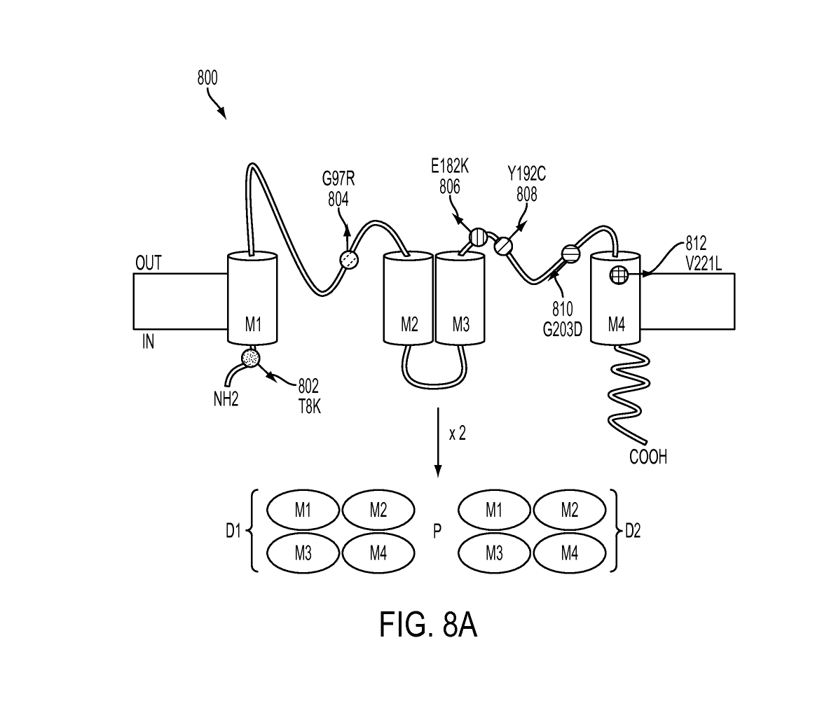

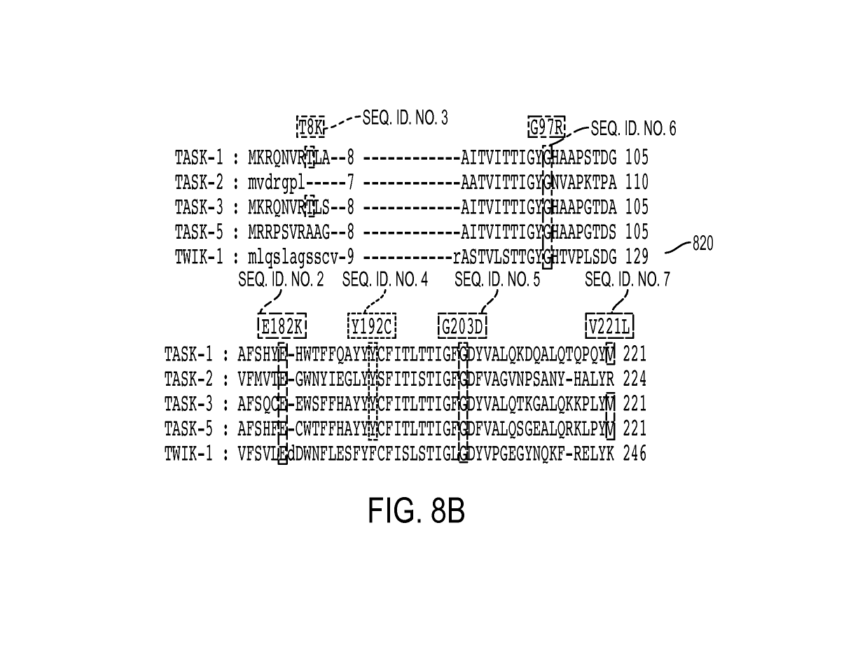

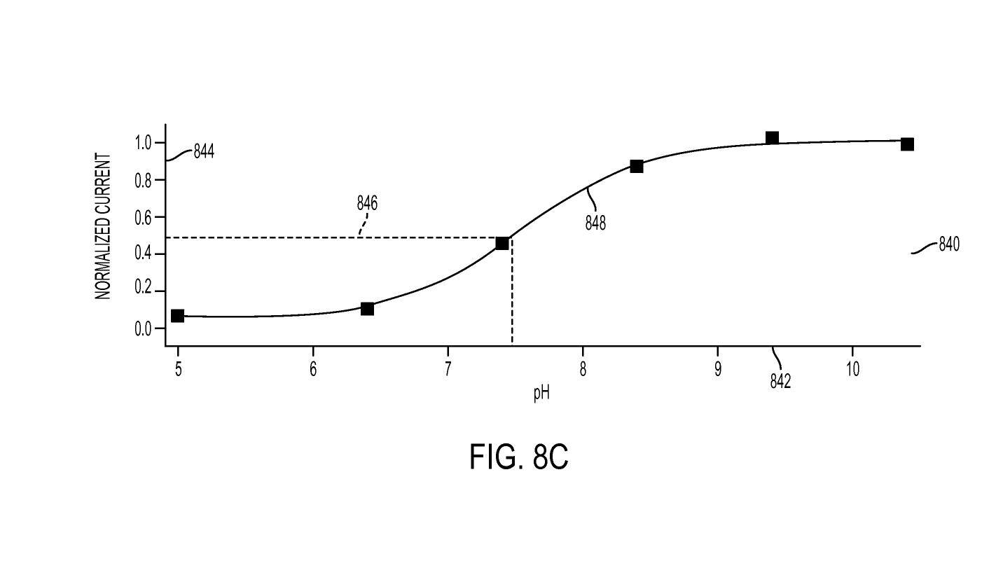

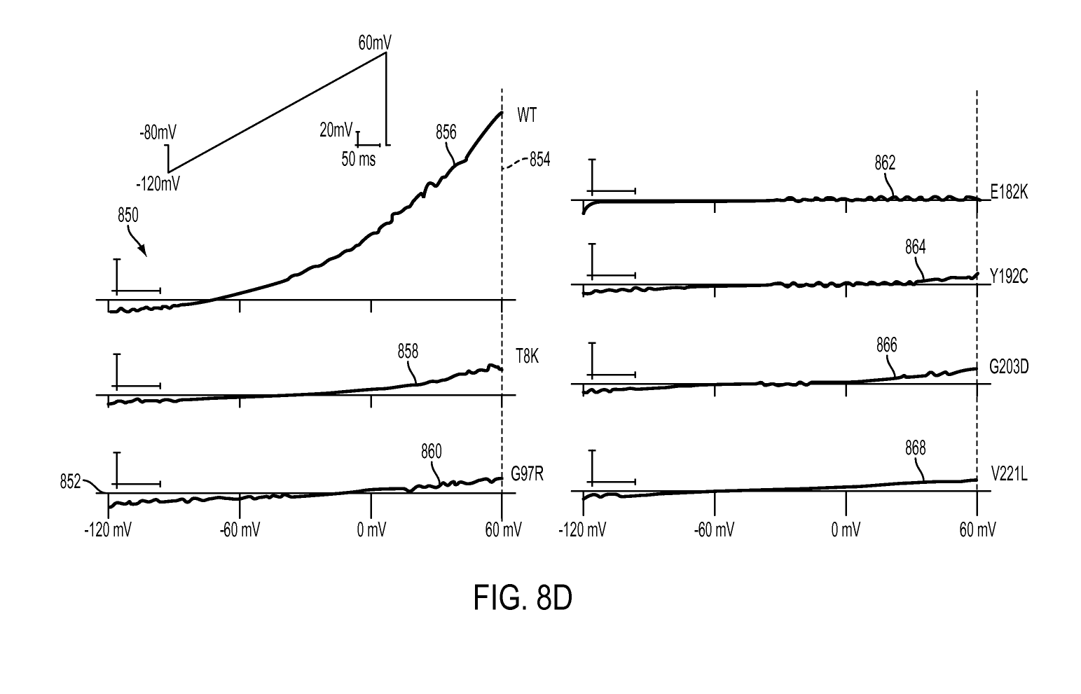

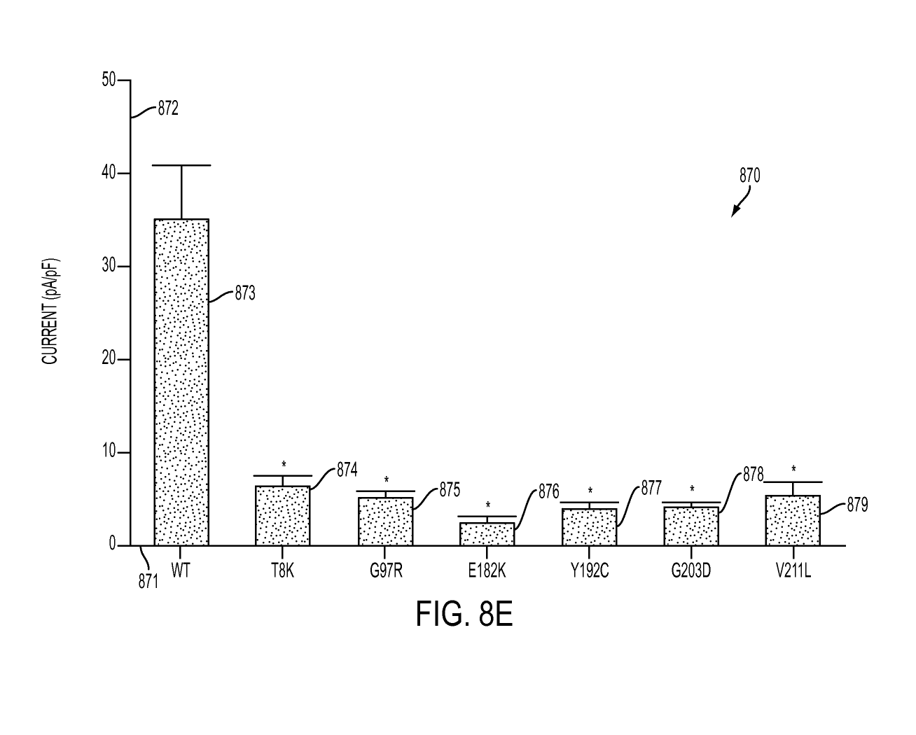

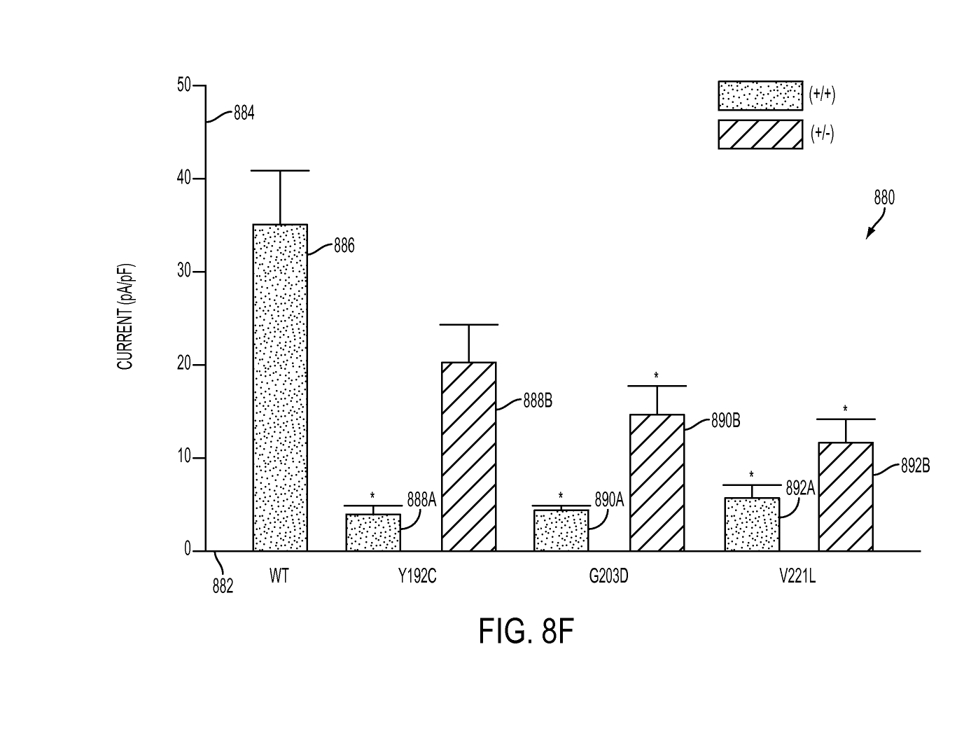

FIG. 8A is a diagram 800 that shows the topology of hTASK-1 channel and TWIK-1 channel and the positions of mutation T8K 802, mutation G97R 804, mutation E182K 806, mutation Y192C 808, mutation G203D 810, and mutation V221L 812. The T8K mutation 802, is in the N-terminus, a part of the channel important to membrane transport out of the endoplasmic reticulum through interaction with 14-3-3 proteins.²⁵ Four of the mutations (G97R 804, E182K 806, Y192C 808, and G203D 810) fall in the pore domains in KCNK3 that are critical for the pH sensitivity and K+ selectivity of this potassium channel family.^{26,27} G97R 804 and G203D 810 are in the pore domain GxG triplet selectivity filters of KCNK3 and may have their deleterious effects as a result of alterations in potassium selectivity. The last mutation V221L 812 falls in one of the transmembrane domains which have been implicated by structural models as important for dimerization.^{17,27}

FIG. 8B is diagram 820 that illustrates the alignment of KCNK3 channel family and TWIK-1 channel and the location of the mutation positions are indicated in the sequences as E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6) and V221L (SEQ. ID. NO. 7). Alignment of the KCNK3 channel with other two-pore domain potassium channels revealed that all of the mutations found here occur at conserved residues likely to be critical for function. Mutations located in conserved residues generally have a deleterious impact on channel function, but the different locations of the mutations suggest that the common channel phenotype may be due to different underlying mechanisms. To investigate the consequences of KCNK3 variants, the impact of the discovered mutations on the hKCNK3 channel was studied. The genetic studies identified six KCNK3 mutations linked to PAH. Each of these mutations was engineered into the hKCNK3 cDNA to study the functional consequences and to determine whether a common channel phenotype might underlie the clinical pathology.

FIG. 8C is a graph 840 that illustrates example wild type (WT) pH dependence of TWIK-1 channel current, according to an embodiment. FIG. 8C includes horizontal axis 842 that indicates physiological pH and vertical axis 844 that indicates normalized current in mV. Wild type (WT) and all mutant channels were tested for pH sensitivity to confirm their identity as hKCNK3 channels. The pH dependence of WT hKCNK3 channels is summarized and illustrated. Dashed line 846 indicates current density at pH 7.4. The solid line 848 is the best fit using dose-response curve. All mutants tested resulted in loss-of-function at physiological pH /(pH 8.4) when expressed alone, a condition that simulates homozygous expression in humans.

FIG. 8D are graphs 850 that illustrate example current traces at pH 7.4 WT hKCNK3 and /T8K, G97R, E182K, Y192C, G203D and V221 mutants hKCNK3 channel, according to an embodiment. FIG. 8D includes a horizontal axis 852 that indicates current in mV and vertical axis 854 that indicates current traces at pH 7.4 for WT hKCNK3 856 T8K 858, G97R 860, E182K 862, Y192C 864, G203D 866 and V221 868 mutant KCNK3 channels. For all current traces, the horizontal scale is -20 mV and the vertical scale is 10 pA/pF. Inset: Voltage ramp protocol. Vertical dashed line: current at 60 mV.

FIG. 8E is a bar graph 870 that illustrates the example results illustrated in FIG. 8D, according to an embodiment. FIG. 8E includes horizontal axis 871 that indicates WT hKCNK3 8873 and all mutant channels T8K 874, G97R 875, E182K 876, Y192 877, G203D 878 and V221 879 tested and vertical axis 872 that indicates normalized current in pA/pF. For every bar 7 to 25 cells were studied. Data are shown as mean+/−SEM.

FIG. 8F is a bar graph 880 that illustrates an example comparison between homozygous and heterozygous expression of WT hKCNK3 with Y192C, G203D and V221 mutants, according to an embodiment. FIG. 8F includes horizontal axis 882 that indicates WT hKCNK3 886 and mutant channels, Y192C 888A, 888B, G203D 890A, 890B and V221 892A, 894B tested and vertical axis 872 that indicates normalized current in pA/pF. Because two pore domain potassium channels assemble as dimers and patients carrying mutations are homozygous, WT and mutant hKCNK3 channel CDNA were co-expressed by transient transfection in COS-7 cells to estimate and approximate expression in heterozygous patients in vivo. Three mutations located in distinct regions of the channel were chosen and in some embodiments it was found that the mutations studied Y192C 888A, 888B, G203D 890A, 890B, and V221L 892A, 892B reduced current density when co-expressed with WT channels compared to expression of WT hKCNK3 channels alone.

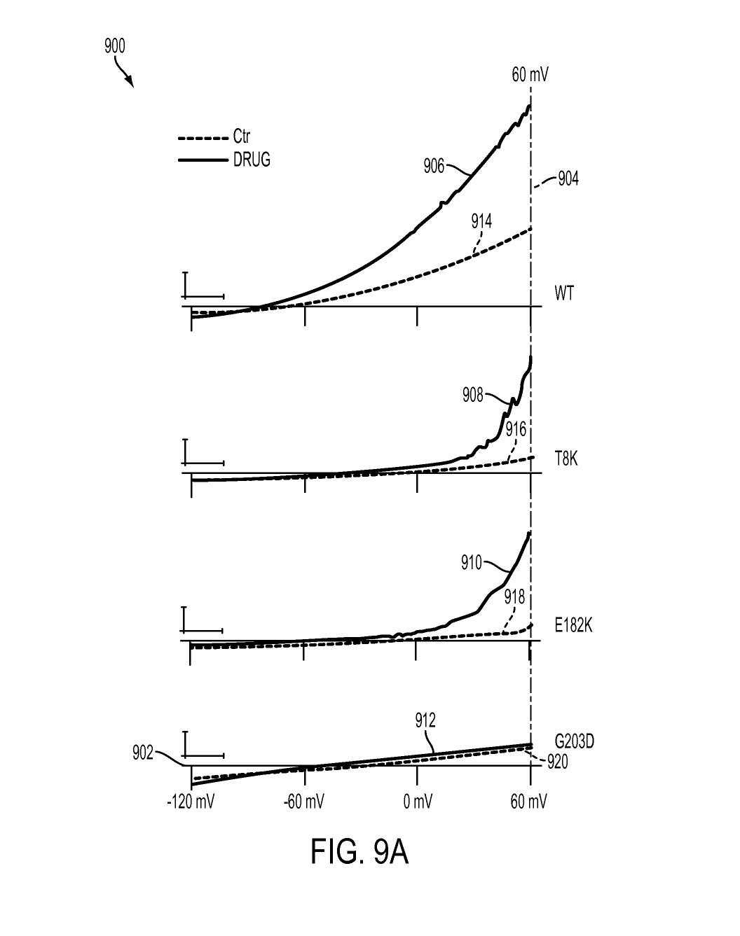

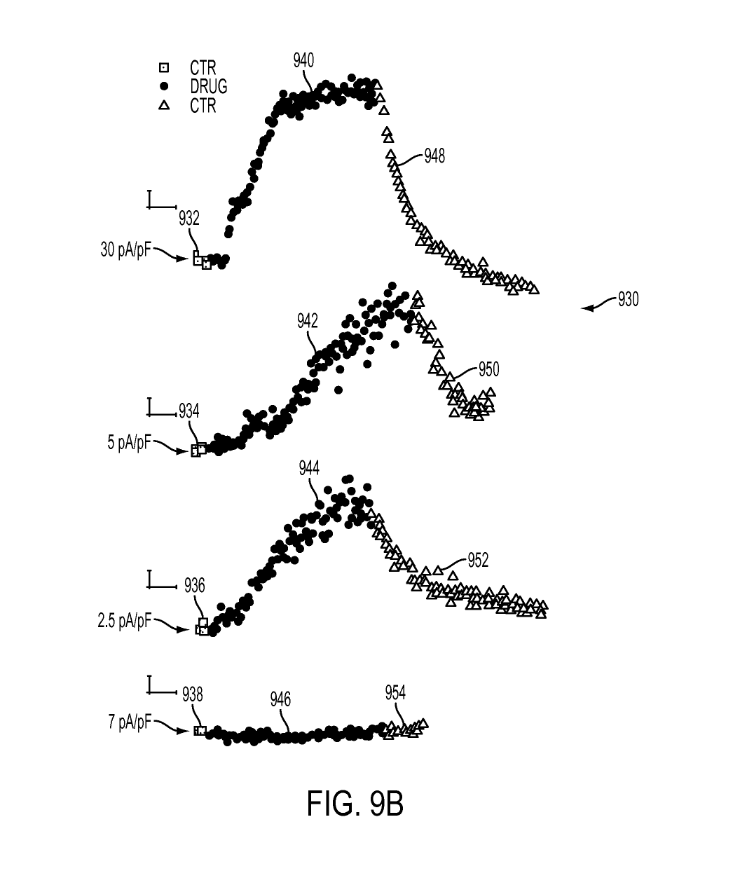

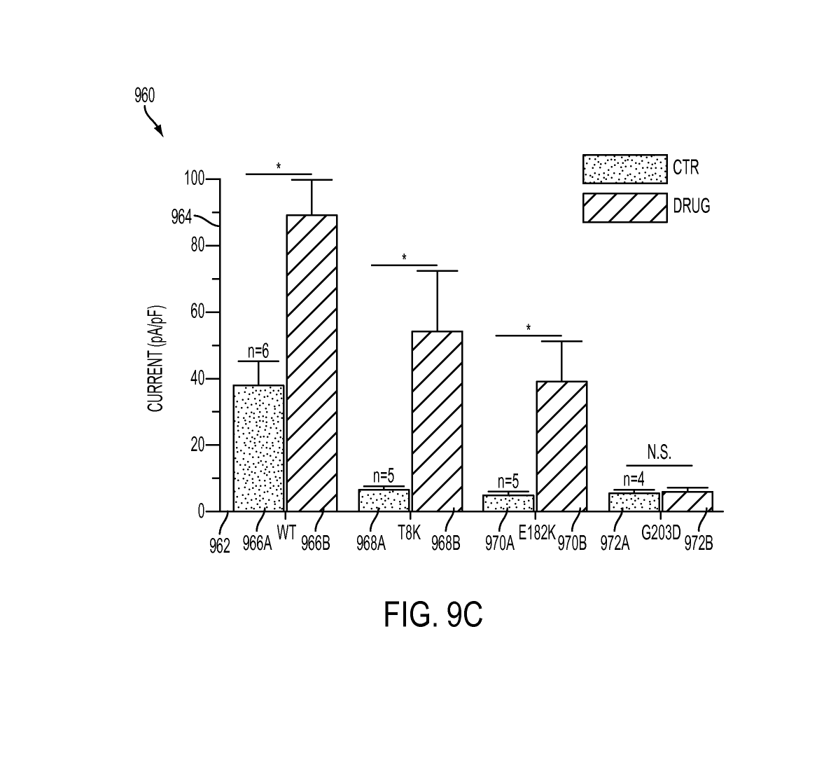

FIG. 9A is a graph 900 that illustrates an example pharmacology recovery of mutant KCNK3 channels where representative recordings show before and after ONO RS-082 application, according to an embodiment. FIG. 9A includes horizontal axes 902 that indicates current in mV and vertical axis 904 that indicates current density at +60 mV. Shown in FIG. 9A are examples of current records in the absence and presence of the drug phospholipase inhibitor ONO RS-082 (10 μM) for WT 914 and 906 respectively, and mutant hKCNK3 channels T8K 916 and 908 respectively, E182K, 918 and 910 respectively, and G203D 920 and 912 respectively as well as current density (at +60 mV) before, during, and after application of ONO-RS-082. Note that not all mutant channels were rescued: G203D mutant channels 912 were not recovered.

FIG. 9B is a graph 930 that illustrates an example time-course of drug application, before for WT 932, T8K 934, E182K 936, and G203D 938 respectively, during for WT 948, T8K 950, E182K 952, and G203D 954 respectively and after drug application for WT 932, T8K 934, E182K 936, and G203D 938 respectively, according to an embodiment.

FIG. 9C is a graph 960 that illustrates an example summary of results of drug effects on the KCNK3 channel before and maximal drug response respectively, according to an embodiment. FIG. 9C includes horizontal axis 962 that indicates WT hKCNK3 channel and mutant T8K, E182K, and G203D channels and vertical axis 964 that indicates current in pA/pF. 966A, 968A, 970A, and 972A represent before drug response for WT hKNK3, T8K, E182K, and G203D respectively and 966B, 968B, 970B, and 972B represent maximal drug response for WT hKNK3, T8K, E182K, and G203D respectively. The results indicate a robust increase in WT hKCNK3 current after application of ONO RS-082 and an increase in current density to near WT 966 control levels for the T8K 968 and E182K 970 mutant channels.

### 2.1. Method

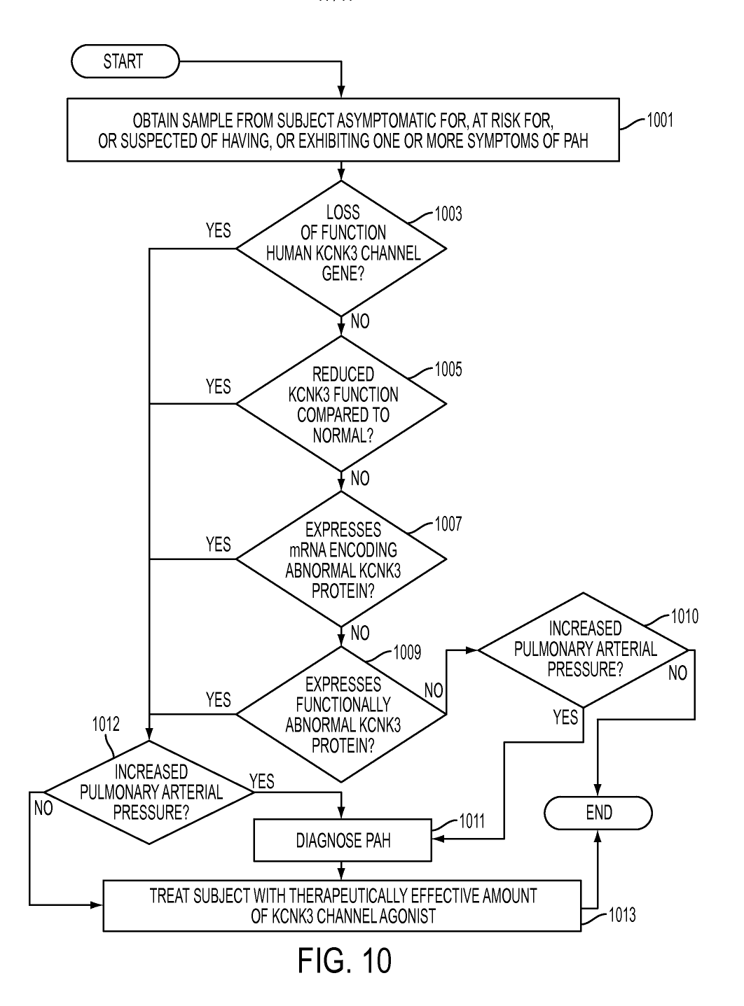

FIG. 10 is a flow chart that illustrates an example method for diagnosing and treating a subject having PAH, according to an embodiment. Although steps are shown in FIG. 10 as integral blocks in a particular order for purposes of illustration, in other embodiments one or more steps or portions thereof may be performed in a different order, or overlapping in time, in series or in parallel, or one or more steps or portions thereof may be omitted, or additional steps added, or the process may be changed in some combination of ways.

In step 1001, a biological sample is obtained from a subject at risk for or suspected of having PAH or has exhibited one ore more symptoms of PAH. In step 1003, it is determined if the biological sample comprises a loss-of-function human KCNK3 channel gene having a gene accession number NM 002246. If so, and it is determined that the subject has increased pulmonary arterial pressure, 1012, then the subject is diagnosed with PAH, as described below with reference to step 1011.

If not, then in step 1005, it is determined if the biological sample has significantly reduced KCNK3 function compared to a biological sample from a normal sample. If so, and it is determined in step 1012 that the subject has increased pulmonary arterial pressure, the subject is diagnosed with PAH in step 1011.

If not, in step 1007, it is determined if the biological sample expresses an mRNA encoding an abnormal form of KCNK3 protein and it is determined in step 1012 that the subject has increased pulmonary arterial pressure,. If so, the subject is diagnosed with PAH in step 1011.

If not, in step 1009, it is determined if the biological sample expresses a functionally abnormal KCNK3 channel protein. If so and it is determined in step 1012 that the subject has increased pulmonary arterial pressure, then it is possible to diagnose the subject as having PAH in step 1011.

If not, but it is determined in step 1010 that the subject has increased pulmonary arterial pressure, then the subject is still diagnosed with having PAH in step 1011. Otherwise, the method ends. In some embodiments, step 1010 is omitted and if no KCNK3 abnormality is detected the process ends without treatment with an KCNK3 channel agonist.

If the subject is diagnosed as having PAH in step 1011, or one of the KCNK3 abnormalities is detected even without a diagnosis of PAH, then the subject is treated in step 1013 by administering a therapeutically effective amount of a KCNK3 channel agonist.

### 2.2. Summary of Experimental Results

Experimental results obtained in various embodiments demonstrate the following.

Six (6) variants of the KCNK3 ion channel were newly discovered for PAH. Three variants (V221L, G203D, and G97R) were identified in familial PAH, and three variants (T8K, E182K and Y192C) were identified in idiopathic PAH.

Each mutation identified falls in highly conserved regions of KCNK3 (FIG. 8), indicating these residues are important for the normal biophysical properties of KCNK3 channel. The molecular mechanisms for loss-of-function are likely diverse based on their location in the channel. The T8K, is in the N- terminus, a part of the channel important to membrane transport out of the endoplasmic reticulum through interaction with 14-3-3 proteins.²⁵ Four of the mutations (G97R, E182K, Y192C, G203D) fall in the pore domains in KCNK3 that are critical for the pH sensitivity and K⁺ selectivity of this potassium channel family.^{26, 27} G97R and G203D, are in the pore domain GxG triplet selectivity filters of KCNK3 (FIG. 8A) and may have their deleterious effects as a result of alterations in potassium selectivity. The last mutation V221L falls in one of the transmembrane domains which have been implicated by structural models as important for dimerization.^{17,27}

All six of the mutations identified resulted in loss-of-function of the KCNK3 ion channel.

Application of the KCNK3 agonist ONO RS-082 produced a robust increase in WT hKCNK3 current and an increase in current density to near WT control levels for two of the 3 mutant channels tested. Recovery of the potassium current was found for T8K and E182K that are associated with idiopathic PAH, but not for the G203D disease-causing mutant associated with familial PAH. Because there are multiple distinct regions of the KCNK3 channel that bind agonists, and because the mutations occur in various critical areas of the KCNK3 molecule, it is not surprising that a single agonist will not affect recovery of function in every mutation. Routine experimentation will show which of the KCNK3 agonists will work to effect recovery in the various mutations.

The KCNK3 ion channel is a therapeutic target. PAH associated with reduced function or loss-of-function of KCNK3 or an abnormal form of KCNK3 channel protein (e.g., due to but limited to reduced potassium channel current at physiological pH, abnormal function, or significantly reduced K+ channel current, abnormal topology based on the crystal structure of TWIK-1 channel, or reduced K+ selectivity) can be treated with one or more KCNK3 agonists (e.g., ONO-RS-82, Treprostinil, Isoflurane, and Halothane). The electrophysiological studies showed that each of the variants identified are loss-of-function mutations. KCNK3 channels are not voltage dependent and are open at negative potentials, thus these mutations likely cause resting membrane potential depolarization which could lead to vasoconstriction in pulmonary artery smooth muscle cells.²⁴ Moreover, these heterozygous missense mutations in KCNK3 act as loss-of-function alleles that lead to increased pulmonary arterial tone, ultimately leading to PAH. The findings in this study parallel findings that Kv channels in human pulmonary artery smooth muscle cells (hPASMCs) down regulate voltage-gated Kv channels in hypertension that have been implicated in altered smooth muscle contraction and proliferation.²⁸

The results herein identified naturally-occurring loss-of-function pathologic mutations in KCNK3 causing both familial and idiopathic PAH, and provide evidence that a pharmacological manipulation of KCNK3 channels with agonists effect a functional recovery of current in PAH-associated mutant channels. Channel responses vary with the location and type of mutation.

Without being bound by theory, variation in KCNK3 function may be a more broadly applicable risk factor for PAH (or a secondary disease modifier) among those with idiopathic PAH or other types of pulmonary hypertension. There is precedent for this concept in PAH, as BMPR2 expression is reduced in the lungs of idiopathic PAH patients without BMPR2 mutations.³⁷ Previous studies of Kv channels support the concept that altered expression and/or function of Kv channels exists in idiopathic PAH patients.³⁸ The major physiological role of the KCNK3 channel is to hyperpolarize smooth muscle cell resting potentials ¹⁴⁻¹⁶ and thus increasing KCNK3 channel activity pharmacologically can hyperpolarize and thus relax cells in which this pathway is impaired, not only by mutation, but also by altered or abnormal cell signaling. This is supported by a recent PAH study using mice with wild-type Kv channels demonstrated the utility of therapeutic Kv channel activation in the treatment of established murine PAH in the absence of known genetic variations in Kv channels.³⁹ Thus, therapeutically targeting KCNK3 may be applicable for PAH patients with increased vascular tone independent of their KCNK3 genetic status.

Identifying genetically at-risk individuals who are asymptomatic (e.g., due to a confirmed gene mutation in the family) provides a potential opportunity for early intervention and treatment of PAH. Given family history, it may be determined whether the subject has a loss-of-function variant of the KCNK3 channel gene (i.e., G203C, G97R, V221L, T8K, E182K, and Y192C). Treatment may be provided prophylactically to these at-risk subjects by administering a therapeutically effective amount of an agonist as described in the methods herein.

In summary, six new mutations in the potassium channel KCNK3 have been identified in both familial and idiopathic PAH patients that facilitate diagnosis and treatment of the disease. The results herein provide novel insight into disease pathogenesis and a novel target for future therapeutic approaches.

### 3. EXAMPLES

### Example 1

### Materials and Methods

Specific materials and methods used in many embodiments are described as set forth below.

### Study Participants and DNA Sample Preparation

Written informed consent for genetic studies was obtained under protocols approved by the institutional review boards (IRBs) at Columbia University Medical Center, Vanderbilt University, or Comité de Protection des Personnes IIe de France VI (ID RCB 2007 AO-1347-46). The diagnosis of PAH was confirmed by medical record review and right heart catheterization. Subjects in this study had been previously sequenced and do not carry BMPR2, ALK1, ENG, SMAD9, or CAV1 mutations. DNA was extracted from peripheral blood leukocytes using Puregene reagents (Gentra Systems Inc., Minnesota, USA).

### Whole Genome Exome Sequencing

Three affected family members were genetically compared by WES assuming an autosomal dominant mode of inheritance. The WES library was prepared with the Agilent SureSelect Paired-End Version 2.0 Human Exome Kit (Agilent, Santa Clara, Calif.). Sequencing of post-enrichment shotgun libraries was performed on an Illumina Genome Analyzer II following manufacturer's protocol for 50 by paired-end reads. The analysis pipeline included genome alignment with BWA (Burrows-Wheeler Aligner) allowing for a 4% error rate (mismatches and gaps) followed by SNP/indel variant analysis.⁴⁰ GATK was used to refine local alignment of reads, recalibrate base quality score, and call variants (SNVs/indels) within targeted regions. SNVs were further filtered using recommended parameters by GATK.⁴¹

### Analysis of SNP Variants

WES data were filtered using dbSNP build (129) on the human assembly hg19, 1000 genome, and NHLBI Exome Sequencing Project (ESP) Exome Variant Server (Available on the World Wide Web in subdomain evs of subdomain gs of domain Washington at category edu in folder EUS) to remove polymorphisms with an allele frequency of >1% in any of the databases. Novel variants present in all three affected family members were analyzed for effect on the amino acid sequence, assigned a coverage-dependent Phred-scaled mutation probability, and analyzed for predicted effect on the protein using SIFT (Available on the World Wide Web in subdomain sift of domain jcvi at category org), PolyPhen-2 (Available on the World Wide Web in subdomain genetics of subdomain bwh of domain harvard at category edu in folder pph2), SeattleSeq Annotation (Available on the World Wide Web in subdomain snp of subdomain gs of domain washington at category edu in folder SeattleSeqAnnotation), mutation taster (Available on the World Wide Web at domain mutationtaster at category org), SNAP (Available on the World Wide Web in domain rostlab at category org in folder services in file name SNAP) and PMut (Available on the World Wide Web at subdomain mmb2 of subdomain pcb of domain ub at category es in folder 8080 in file name PMut). Functions of candidate genes were evaluated through GeneDistiller 2, Online Mendelian Inheritance in Man database (OMIM), and the published literature. Alignment of the KCNK3 (AKA KCNK3) protein sequence was performed with the use of the ClustalW program.

### Segregation Analysis and Sanger Sequencing

PCR primers were designed flanking the variant identified in KCNK3 and directly sequenced in all available members of the study family. Capillary sequence reads were analyzed using Sequencher software (GeneCodes Inc.). To identify additional mutations, primers were designed to amplify all coding regions and at least 20 by of adjacent intronic sequence of KCNK3. DNA samples from 82 unrelated familial and 230 idiopathic pulmonary hypertension patients were sequenced to attempt to replicate the findings in the initial family and determine the frequency of mutations in KCNK3 in hereditary and idiopathic PAH. Sequence variants were confirmed with bidirectional sequencing. Population allele frequencies for all variants were assessed in dbSNP, 1000 genomes and NHLBI exome variant server databases. Only novel variants were considered as potentially pathological for disease. For familial PAH patients with KCNK3 mutations, available family members were tested to evaluate segregation within the family.

### Lung Tissue Sampling

Lung tissue was obtained from explanted lungs of two idiopathic PAH patients. The specimen was fixed in 10% formalin, processed, embedded in paraffin, sectioned and stained with hematoxylin-eosin, CD31, alpha smooth muscle actin (SMA), or von Willebrand factor (vWF), and Verhoeff-Van Gieson (VVG)Elastic Staining.

Expression and Function Analysis of hKCNK3 Channels

Functional analysis of the KCNK3 channel was performed to evaluate the functional effects of the rare genetic variants identified and to provide insight into the disease mechanism. pIRES-CD8-hKCNK3 was kindly provided by Florian Lesage (Institut de Pharmacologie Moléculaire et Cellulaire, Centre National de la Recherche Scientifique and Université de Nice Sophia Antipolis, Valbonne, France). hKCNK3 DNA was amplified and inserted into pcDNA 3.1 (+) vector (Invitrogen). Site-directed mutagenesis was performed using the QuikChange kit (Stratagene) and all constructs were sequenced before use. COS-7 cells (American Type Culture Collection) were grown under conventional conditions (5% CO₂, DMEM (Gibco) +GlutaMax medium (Gibco) and 10% fetal bovine serum (Gibco)). Cells were transfected with lipofectamine (Invitrogen) as previously described.⁴² To approximate homozygote activity cells were transfected with 2 μg total hKCNK3 cDNA (either WT, or mutant). To approximate heterozygote activity, cells were transfected with WT (1.5 μg) plus mutant hKCNK3 (1.5 μg) cDNA. In both cases GFP (Clontech, 1 μg) was also included in transfections. The topology of the KCNK3 channel was based on the crystal structure of TWIK-1 channel⁴³ and alignment was performed using GeneDoc.⁴⁴ Membrane currents were measured with whole-cell patch-clamp procedures using Axopatch 200B amplifiers (Axon Instruments). The following solutions (mmol/L) were used: internal 150 KCl, 3 MgCl₂, 5 EGTA and 10 HEPES at pH 7.2 with KOH; and external 150 NaCl, 5 KCl, 1 mM MgCl₂, 1.8 CaCl₂ and 10 mM HEPES at pH 7.4. Voltage ramp protocols (−120 mV to +60 mV, 360 mV/s) were imposed once every 3 sec from a -80 mV holding potential to measure expressed K⁺ channel currents. Current measured at +60 mV was selected for comparison of constructs and drug actions. The KCNK3 channel is well-known to be pH sensitive⁴⁵ and all expressed currents were tested for pH-sensitivity, but comparisons of the effects of mutations and drugs were carried out at physiological pH (pH 7.4). In all analysis and figures whole cell current is shown normalized to cell capacitance. ONO RS-082 (Enzo Life Sciences) was dissolved in DMSO (10 mM stock solution) and diluted to 10 μM in both drug-free (control) and drug-containing external solutions. Analysis was carried out in Origin 7.0 (Microcal Software, Northampton, Mass.). Analyzed data are shown as mean+/−S.E.M. Statistical significance was determined using one-way ANOVA. p<0.05 was considered statistically significant.

### Patient Diagnosis

The diagnosis of PAH was confirmed by medical record review and right heart catheterization, and subjects do not carry BMPR2, ALK1, ENG, SMAD9, or CAV1 mutations. To summarize, three affected family members were genetically compared by WES assuming an autosomal dominant mode of inheritance, and variants were filtered based upon allele frequency in controls and predicted pathogenicity. Variants identified in KCNK3 channels were confirmed by Sanger sequencing in all available members of the study family.

To identify additional mutations and mutation carriers, DNA samples from 82 unrelated familial and 230 idiopathic pulmonary hypertension patients were sequenced and WES from 10 additional familial cases were reviewed to replicate the findings in the initial family and determine the frequency of mutations in KCNK3 in hereditary and idiopathic PAH. For familial PAH patients with KCNK3 mutations, available family members were tested to evaluate segregation within the family. Functional analysis of the hKCNK3 channel was carried out to evaluate the genetic variants identified. Mutations were engineered into hKCNK3 and expressed using transient transfection in COS-7 cells. Whole patch clamp procedures were used to measure expressed currents and their response to pH and pharmacological agents.

### Example 2

### WES Results

The average read depth of the WES was 78.7, with 87.5% of the target region having greater than 20X sequencing depth. Variants in dbSNP, 1000 genomes and NHLBI exome variant server databases with an allele frequency of >1% were removed, leaving 4719 rare or novel variants present in any of the three affected individuals. Heterozygous variants shared by the three affected individuals were filtered, and 377 novel SNVs and 6 indels were left. Because the pedigree suggested an autosomal dominant mode of inheritance, homozygous variants were excluded. Variants were then filtered for predicted pathogenic effects on the protein. Indels, nonsense variants, splice sites alternation and missense variants predicted to be damaging or unknown by POLYPHEN2 were prioritized, leaving175 heterozygous SNVs and 5 indels carried by every affected family member. 80 SNVs had a score of “pass” evaluated by GATK filterflag.¹³ Variants were predicted to be deleterious by either SIFT or SeattleSeq SNP Annotation. Of these 19 single nucleotide variants and 5 indels, the novel missense variant c.608 G>A, G203D in KCNK3 was identified as the strongest candidate as a PAH-causing mutation because KCNK3 of this channel's importance in the regulation of pulmonary vascular tone in human.¹⁴ According to the homology modeling of hKCNK3, amino acid G203 is located in the second pore region, which is critical for gating of potassium channel. Examination of SNP database indicated that there are no common variants in KCNK3.

### Example 3

### Gene Mutation Confirmation in the Family

Segregation analysis was performed on all available family members (FIG. 1, FIG. 2, and FIG. 3). II.3 in the pedigree did not carry the c.608 G>A, G203D in KCNK3 novel variant, indicating that the affected II.2 is an obligate carrier. III.2 and III.5 carry the c.608 G>A, G203D in KCNK3. Although III.5 was initially unaffected when initially recruited, he has subsequently developed PAH. III.2 remains unaffected (FIG. 1). One hundred ethnically matched unrelated unaffected control Caucasian DNA samples were genotyped, and the c.608 G>A KCNK3 variant was not present in any of the 100 controls. The c.608 G>A, G203D KCNK3 variant is predicted to be damaging by SIFT and POLYPHEN2 and is located in the highly conserved second potassium channel pore region.

### Example 4

### Case Series of PAH Patients

Ten additional probands with familial PAH were studied by WES. Two heterozygous novel KCNK3 variants G97R and V221L were identified in two of these families. These variants were confirmed by Sanger sequencing and tested in available family members and segregated with disease (FIG. 2 and FIG. 3). We screened an additional 82 unrelated probands with familial PAH and 230 patients with idiopathic PAH for mutations in KCNK3. Three novel heterozygous amino acid substitutions T8K, E182K and Y192C were identified among the 230 idiopathic PAH patients. The six variants are all predicted to be damaging. The frequency of KCNK3 mutations in our idiopathic PAH cases is 1.30% (3/230) and 3.2% in familial cases (3/93).

### Clinical Phenotypes of Mutation Carriers

Familial PAH pedigrees are shown (FIG. 1, FIG. 2, and FIG. 3), and there is evidence of incomplete penetrance. Age of diagnosis ranged from age 8 to 44 (Table 1). Males and females were affected. No patient was responsive to acute vasodilator challenge. Three patients ultimately required lung transplant.

TABLE 1
Clinical information of PAH patients with KCNK3 mutations.
Patients FPPH050 III.1 and II.2, PSL3555 III.1, and TNN0011 are familial PAH patients also shown in
FIG. 1, FIG. 2, and FIG. 3. SPH253 JM1188, SPH400 LP1959, and JM0041 are idiopathic PAH patients.

| | FPPH050 | FPPH050 | PSL3555 | TNN0011 | SPH253 | SPH400 | |
|---|---|---|---|---|---|---|---|
| | III.1 | II.2 | III.1 | III.2 | JM1188 | LP1959 | JM0041 |
| Gender | Female | Female | Male | Female | Male | Female | Male |
| Age at Diagnosis, yrs | 37 | 44 | 19 | 29 | 25 | 38 | 8 |
| Current age, yrs | Deceased | Deceased | 20 | Deceased | 40 | 43 | 20 |
| Lung Transplant | No | No | No | At age 33 | At age 29 | No | At age 15 |
| KCNK3 Variant | G203D | G203D | G97R | V221L | E182K | T8K | Y192C |
| PAH type | Familial | Familial | Familial | Familial | Idiopathic | Idiopathic | Idiopathic |
| Mean Pulmonary | 62 | 76 | 86 | 67 | 101 | 54 | 107 |
| Artery Pressure at | | | | | | | |
| Diagnosis (mmHg) | | | | | | | |
| Right Atrial Pressure | 7 | 18 | 11 | 7 | 16 | 24 | 3 |
| at Diagnosis (mmHg) | | | | | | | |
| PCWP (mmHg) | 10 | 13 | 13 | 7 | 15 | 6 | 10 |
| CI (L/min/m2) | 3.16 | 2.74 | 3.22 | 2.72 | 1.73 | 1.21 | 2.70 |
| PVR index (dynes · sec · | 1,316 | 1,839 | 1,813 | 1,764 | 3,977 | 3,174 | 2,874 |
| cm−5/m2) | | | | | | | |
| NYHA Functional Class, | N/A | N/A | III | III | III | III | III |
| at Diagnosis | | | | | | | |
| Response to Acute | N/A | N/A | No | No | No | No | No |
| Vasodilator Challenge | | | | | | | |
| Arrhythmias | | Partial | | | Atrial | First | |
| | | RBBB | | | flutter | Degree AV | |
| | | | | | | Block; RBBB | |

PAH: Pulmonary arterial hypertension
PCWP: Pulmonary capillary wedge pressure
CI: Cardiac index
PVR: Pulmonary vascular resistance
NYHA: New York Heart Association
AV: atrioventricular
RBBB: right bundle branch block

The histopathology is shown in FIG. 3, FIG. 4, FIG. 5, FIG. 6, and FIG. 7 and demonstrates hypertrophy of the media of muscular pulmonary arteries, progressive generalized arterial dilatation with formation of complex plexiform lesions.

### Example 5

### Loss of Function of hKCNK3 Channel is Associated with PAH

Alignment of the KCNK3 channel with other two-pore domain potassium channels revealed that all of the mutations found here occur at conserved residues likely to be critical for function (FIG. 8A, FIG. 8B). To investigate the consequences of KCNK3 variants, the impact of the discovered mutations on the hKCNK3 channel was studied. The genetic studies identified six KCNK3 mutations linked to PAH. Each of these mutations was engineered into the hKCNK3 cDNA to study the functional consequences and to determine whether a common channel phenotype might underlie the clinical pathology. Wild type (WT) and all mutant channels were tested for pH sensitivity to confirm their identity as hKCNK3 channels. The pH dependence of WT hKCNK3 channels is summarized and illustrated in FIG. 5. All mutants tested resulted in loss-of-function at physiological pH (pH 7.4) when expressed alone, a condition that simulates homozygous expression in humans. However, because two pore domain potassium channels assemble as dimers¹⁷ and patients carrying mutations are heterozygous, WT and mutant hKCNK3 channel cDNA were co-expressed by transient transfection in COS-7 cells to estimate/approximate expression in heterozygous patients in vivo. For these experiments 3 mutations located in distinct regions of the channel were chosen and it was found that the mutations studied (Y192C, G203D and V221L) reduced current density when co-expressed with WT channels compared to expression of WT hKCNK3 channels alone (FIG. 8F).

### Example 6

### KCNK3 Channel Agonist Rescues Ion Channel Function

Experiments were conducted showing that ONO RS-082 was capable of rescuing ion channel activity in three mutant hKCNK3 mutant channels: T8K, E182K and G203D. Recovery of the potassium current was found for T8K and E182K, but not for G203D disease-causing mutants. Because there are multiple distinct regions of the KCNK3 channel that can bind agonists, and because the mutations are all in critical areas of the molecule, it is not surprising that a single agonist will not affect recovery of function in every mutation. Routine experimentation will show which of the KCNK3 agonists will work to effect recovery in the various mutations.

Shown in FIG. 9 are examples of current recordings in the absence and presence of the phospholipase inhibitor ONO RS-082 (10 μM) for WT and mutant hKCNK3 channels (FIG. 9A), as well as current density (at +60 mV) before, during and after application of the drug (FIG. 9B). Steady state effects of the drug are summarized in FIG. 9C. The results indicate a robust increase in WT hKCNK3 current after application of ONO RS-082 and an increase in current density to near WT control levels for the T8K and E182K mutant channels.

### 4. ALTERNATIVE EMBODIMENTS

### Pharmaceutical Formulations

In alternative embodiments, pharmaceutical formulations involve administration of pharmaceutical compositions and formulations which include one or more KCNK3 agonists (hereafter “therapeutic agents”), including ONO RS-082, Treprostinil (also known as Remodulin, Tyvaso), Isoflurane and Halothane.

The pharmaceutical compositions of the present invention may be administered in a number of ways including oral, local intravenous, parenteral/intra-arterial, subcutaneous, intraperitoneal or intramuscular injection or infusion; or intracranial, e.g., intrathecal or intraventricular, administration. Pharmaceutical compositions of the present invention contain the therapeutic in an amount sufficient to prevent or treat PAH or other disease or condition associated with significantly reduced KCNK3 channel function (e.g., central sleep apnea and prosopagnosia) compared to normal levels, (hereafter collectively “an enumerated disease”).

The therapeutic agent can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

New pharmaceutical formulations according to embodiments of the invention include combinations of two or more therapeutic agents (KCNK3 agonists).

A suitable subject can be an individual or animal that is suspected of having, has been diagnosed as having, or is at risk of developing an enumerated disease, and like conditions as can be determined by one knowledgeable in the art.

Techniques for formulation and administration can be found in “Remington: The Science and Practice of Pharmacy” (20th edition, Gennaro (ed.) and Gennaro, Lippincott, Williams & Wilkins, 2000), incorporated herein by reference. The pharmaceutical compositions of the present invention can be administered to the subject by a medical device, such as, but not limited to, catheters, balloons, implantable devices, biodegradable implants, prostheses, grafts, sutures, patches, shunts, or stents. A detailed description of pharmaceutical formulations of oligonucleotides is set forth in U.S. Pat. No. 7,563,884.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients. Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

The active therapeutic agents (KCNK3 agonists) can be administered as often as is deemed necessary, including daily at least once, multiple times per day, or less often, and they can be formulated for administration can be via any route as is explained below.

Therapeutic amounts of the KCNK3 agonists of the invention range from about 0.0001 to 100 mg/kg/day. Treprostinil is known since it is presently used to treat PAH. Treprostinil is substantially metabolized by the liver. It has a half life of 4 hours and is excreted in the urine as 4% unchanged drug and 64% as identified metabolites); feces (13%). Treprostinil is indicated for the treatment of pulmonary arterial hypertension in patients with NYHA Class II-IV symptoms to diminish symptoms associated with exercise.^{[1]} It may be administered as a continuous subcutaneous infusion or continuous intravenous infusion; however, because of the risks associated with chronic indwelling central venous catheters, including serious blood stream infections, continuous intravenous infusion should be reserved for patients who are intolerant of the subcutaneous route, or in whom these risks are considered warranted. Treprostinil may be administered as a continuous subcutaneous infusion or continuous intravenous infusion via a small infusion pump that the patient must wear at all times. Treprostinil can be given subcutaneously by continuous infusion using an infusion set connected to an infusion pump, but also may be given intravenously via a central venous catheter if the patient is unable to tolerate subcutaneous administration because of severe site pain or reaction. Remodulin Full Prescribing Information US Patent No. 5,153,222. The pharmacokinetics of continuous subcutaneous treprostinil are linear over the dose range of 1.25 to 125 ng/kg/min (corresponding to plasma concentrations of about 15 pg/mL to 18,250 pg/m) and can be described by a two-compartment model. Dose proportionality at infusion rates greater than 125 ng/kg/min has not been studied.

It is anticipated that similar doses of other KCNK3 agonists can be used as starting points to determine efficacy ranges. TASK-1 is activated by volatile general anesthetics Isoflurane and Halothane. Isoflurane (2-chloro-2-(difluoromethoxy)-1,1,1-trifluoro-ethane) is a halogenated ether used for inhalational anesthesia. Halothane (trademarked as Fluothane) is an inhalational general anesthetic. Its IUPAC name is 2-bromo-2-chloro-1,1,1-trifluoroethane. Patel A J, et al., Inhalational anesthetics activate two-pore-domain background K+ channels, Nat Neurosci. 1999 May; 2(5):422-6.

Factors that affect dose further include the severity of the disorder and the bioavailability The optimum amount and route of administration is to be determined by routine experimentation, for example, by determining the amount needed to reduce one or more symptoms of an enumerated disease, or increase potassium current through a KCNK3 channel in a subject, preferably a human, as can be measured electrophysiologically as described herein.

### Kits

In other embodiments of the invention, various kits are also provided. Typically, the kits include a pharmaceutical composition as described herein and instructions for the use of the pharmaceutical composition and dosage regime. The kit can comprise the pharmaceutical composition of the invention in a suitable container with labeling and instructions for use. The container can be, but is not limited to, a dropper or tube. The pharmaceutical composition of the invention can be filled and packaged into a plastic squeeze bottle or tube. Suitable container-closure systems for packaging pharmaceutical compositions of the invention are commercially available for example, from Wheaton Plastic Products, 1101 Wheaton Avenue, Millville, N.J. 08332.

Preferably, instructions are packaged with the formulations of the invention, for example, a pamphlet or package label. The labeling instructions explain how to administer pharmaceutical compositions of the invention, in an amount and for a period of time sufficient to treat or prevent PAH and symptoms associated therewith. Preferably, the label includes the dosage and administration instructions, the topical formulation's composition, the clinical pharmacology, drug resistance, pharmacokinetics, absorption, bioavailability, and contraindications.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

### Detection of Single Nucleotide Polymorphisms (SNPs)

Methods for detecting SNPs present in a polynucleotide sequence involve procedures that are well known in the art (e.g., amplification of nucleic acids). See, e.g., Single Nucleotide Polymorphisms: Methods and Protocols, Pui-Yan Kwok (ed.), Humana Press, 2003. Although many detection methods employ polymerase chain reaction (PCR) steps to detect SNPs of a polynucleotide, other amplification protocols may also be used including, e.g., ligase chain reactions, strand displacement assays, and transcription-based amplification systems.

In general, detection of SNPs (e.g., KCNK3 channel SNPs) or other polymorphisms can be performed using oligonucleotide primers and/or probes. Oligonucleotides can be prepared by any suitable method (e.g., chemical synthesis). Oligonucleotides can be synthesized using commercially available reagents and instruments. Alternatively, they can be purchased through commercial sources. Methods of synthesizing oligonucleotides are well known in the art (see, e.g., Narang et al., Meth Enzymol. 68: 90-99, 1979 and U.S. Pat. No. 4,458,066). In addition, modifications to such methods of oligonucleotide synthesis may be used, e.g., to impact enzyme behavior with respect to the synthesized oligonucleotides. For example, incorporation of modified phosphodiester linkages (e.g., phosphorothioate, methylphosphonates, phosphoamidate, or boranophosphate) into an oligonucleotide may be used to prevent cleavage of the oligonucleotide at a selected site.

The genotype of an individual for an KCNK3 channel polymorphism can be determined using many detection methods that are well known in the art including, e.g., hybridization using allele-specific oligonucleotides, primer extension, allele-specific ligation, sequencing, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microarrays, fluorescence polarization assays, and matrix-assisted laser desorption ionization (MALDI) mass spectrometry.

Detecting the presence of a SNP is generally performed by analyzing a sample (e.g., a biological sample containing nucleic acid) that is obtained from an individual. Often, the biological sample includes genomic DNA. The genomic DNA is typically obtained from blood samples, but may also be obtained from other cells (e.g., pulmonary artery cells) or tissues (e.g., lung tissue). For example, the biological sample may include cells, protein or membrane extracts of cells, or blood, or biological fluids. Biological samples may be obtained by standard methods including, e.g., venous puncture and surgical biopsy. In certain embodiments, the biological sample is a lung tissue sample obtained by needle or surgical biopsy.

It is also possible to analyze RNA samples for the presence of polymorphic alleles. For example, mRNA can be used to determine the genotype of an individual at one or more KCNK3 channel polymorphic sites. In this case, the biological sample is obtained from cells in which the target nucleic acid is expressed, e.g., pulmonary artery cells. Such an analysis can be performed by first reverse-transcribing the target RNA using, for example, a viral reverse transcriptase, and then amplifying the resulting cDNA or, alternatively, using a combined high-temperature reverse-transcription-polymerase chain reaction (RT-PCR), as described in U.S. Pat. Nos. 5,310,652; 5,322,770; 5,561,058; 5,641,864; and 5,693,517.

Other nucleic acid samples that may be analyzed include, e.g., genomic fragmented DNA, PCR-amplified DNA, and cDNA.

The nucleic acid samples taken from an individual may be compared, for example, to the wild-type nucleic acid sequence of, e.g., KCNK3 channel from a normal subject, described herein.

Frequently used methodologies for the analysis of biological samples to detect SNPs are briefly described. However, any method known in the art can be used in the invention to detect the presence of SNPs.

### Allele-Specific Hybridization

This technique, also referred to as allele-specific oligonucleotide (ASO) hybridization, relies on distinguishing between two DNA molecules differing by one base by hybridizing an oligonucleotide probe that is specific for one of the variants to an amplified product obtained from amplifying the nucleic acid obtained from the biological sample. This method typically employs short oligonucleotides, e.g., oligonucleotides 15-20 bases in length. The oligonucleotide probes are designed to hybridize to one variant, but not to another variant. Hybridization conditions should be sufficiently stringent so that there is a significant difference in hybridization intensity between alleles, whereby an oligonucleotide probe hybridizes to only one of the alleles. The amount and/or presence of an allele may be determined by measuring the amount of allele-specific oligonucleotide that is hybridized to the sample. Typically, the oligonucleotide is labeled (e.g., with a fluorescent label). For example, an allele-specific oligonucleotide may be applied to immobilized oligonucleotides representing FHOD3 SNP sequences. After stringent hybridization and subsequent washing, fluorescence intensity is measured for each SNP oligonucleotide.

According to the invention, SNPs can be identified in a high throughput fashion via a microarray that allows the identification of one or more SNPs at any given time. Such microarrays are described, for example, in WO 00/18960. An array usually involves a solid support on which nucleic acid probes capable of hybridizing to one or more nucleic acid molecules having a SNP in the KCNK3 gene wherein said SNP is selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7). These arrays may be produced using mechanical synthesis methods or light-directed synthesis methods that incorporate a combination of photolithographic methods and solid-phase synthesis methods. (See, for example, Fodor et al., Science 251: 767-777, 1991, and U.S. Pat. Nos. 5,143,854 and 5,424,186, each of which is hereby incorporated by reference.) Although a planar array surface is typically used, the array may be fabricated on a surface of virtually any shape or even a multiplicity of surfaces. Arrays may be nucleic acids on beads, fibers (e.g., fiber optics), glass, or any other appropriate substrate. In one embodiment, the microarray is a beadchip (e.g., a 370CNV Infinium chemistry-based whole genome DNA analysis beadchip (Illumina)).

In one example, SNP arrays utilize ASO hybridization to detect polymorphisms. SNP arrays include immobilized nucleic acid sequences or target sequence, one or more labeled allele-specific oligonucleotide probes, and a detection system that records and interprets the hybridization signal. To achieve relative concentration independence and minimal cross-hybridization, raw sequences and SNPs of multiple databases are scanned to design the probes. Each SNP on the array is interrogated with different probes.

Other suitable assay formats for detecting hybrids formed between probes and target nucleic acid sequences in a sample are known in the art and include the immobilized target (e.g., dot-blot) formats and immobilized probe (e.g., reverse dot-blot or line-blot) assay formats. Dot-blot and reverse dot-blot assay formats are described in U.S. Pat. Nos. 5,310,893; 5,451,512; 5,468,613; and 5,604,099, each incorporated herein by reference.

### Allele-Specific Primers

Polymorphisms are also commonly detected using allele-specific amplification or primer extension methods. These reactions typically involve use of primers that are designed to specifically target a polymorphism via a mismatch at the 3′-end of a primer. The presence of a mismatch affects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product, signifying the particular allelic form is present. Allele-specific amplification- or extension-based methods are described, for example, in WO 93/22456 and in U.S. Pat. Nos. 5,137,806; 5,595,890; 5,639,611; and 4,851,331.

### Detectable Probes

Genotyping can also be performed using a TaqMan (Applied Biosystems) (or 5′-nuclease) assay, as described in U.S. Pat. Nos. 5,210,015; 5,487,972; 5,491,063; 5,571,673; and 5,804,375.

The TaqMan probe principle relies on the 5′.fwdarw.3′ nuclease activity of Taq polymerase to cleave a dual-labeled probe during hybridization to the complementary target sequence and fluorophore-based detection. TaqMan probes consist of a fluorophore covalently attached to the 5′-end of the oligonucleotide probe and a quencher at the 3′-end. Several different fluorophores (e.g., 6-carboxyfluorescein or tetrachlorofluorescin) and quenchers (e.g., tetramethylrhodamine or dihydrocyclopyrroloindole tripeptide) may be used. The quencher molecule quenches the fluorescence emitted by the fluorophore when excited by the cycler's light source via fluorescence resonance energy transfer. As long as the fluorophore and the quencher are in proximity, quenching inhibits a fluorescence signal.

TaqMan probes are designed such that they anneal within a DNA region amplified by a specific set of primers. As the Taq polymerase extends the primer and synthesizes the nascent strand, the 5′.fwdarw.3′ exonuclease activity of the polymerase degrades the probe that has annealed to the template. Degradation of the probe releases the fluorophore from the probe such that the fluorophore and quencher are no longer in close proximity, thus relieving the quenching effect and allowing fluorescence of the fluorophore. Hence, fluorescence detected in, for example, a real-time PCR thermal cycler is directly proportional to the fluorophore released and the amount of DNA template present in the PCR.

The hybridization probe can be an allele-specific probe that discriminates between the SNP alleles. Alternatively, the method can be performed using an allele-specific primer and a labeled probe that binds to amplified product.

Probes detectable upon a secondary structural change are also suitable for detection of a polymorphism, including SNPs. Exemplary secondary structure or stem-loop structure probes include molecular beacons (e.g., Scorpion® primers and probes). Molecular beacon probes are single-stranded oligonucleotide probes that can form a hairpin structure in which a fluorophore and a quencher are usually placed on the opposite ends of the oligonucleotide. At either end of the probe, short complementary sequences allow for the formation of an intramolecular stem, which enables the fluorophore and quencher to come into close proximity The loop portion of the molecular beacon is complementary to a target nucleic acid of interest. Binding of the probe to its target nucleic acid of interest forms a hybrid that results in the opening of the stem loop and a conformational change that moves the fluorophore and the quencher away from each other, leading to a more intense fluorescent signal.

### DNA Sequencing and Single Base Extensions

SNPs can also be detected by direct sequencing. Methods include, e.g., dideoxy sequencing, Maxam-Gilbert sequencing, chain-termination sequencing (e.g., Sanger method), pyrosequencing, Solexa sequencing, SOLiD sequencing, or any other sequencing method known to one of skill in the art.

### Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Polymorphisms may also be detected using capillary electrophoresis. Capillary electrophoresis allows identification of repeats in a particular allele. The application of capillary electrophoresis to the analysis of DNA polymorphisms is well known in the art.

### Single-Strand Conformation Polymorphism Analysis

Alleles of target sequences can be differentiated using single-strand conformation polymorphism analysis, which identifies base differences by alteration in electrophoretic migration of single-stranded PCR products. Amplified PCR products can be generated as described above and heated or otherwise denatured to form single-stranded amplification products. Single-stranded nucleic acids may refold or form secondary structures, which are partially dependent on the base sequence. The different electrophoretic mobilities of single-stranded amplification products can be related to base-sequence differences between alleles of target sequences.

SNP detection methods often employ labeled oligonucleotides. Oligonucleotides can be labeled by incorporating into or onto an oligonucleotide a label detectable by spectroscopic, photochemical, biochemical, immunochemical, or chemical means. Useful labels include fluorescent dyes (e.g., fluorescein, rhodamine, Oregon green, eosin, cyanine derivatives, naphthalene derivatives, coumarin derivatives, oxadiazole derivatives, BODIPY, pyrene derivatives, proflavin, acridine orange, crystal violet, malachite green, Alexa Fluor, porphin, phtalocyanine, bilirubin, DAPI, Hoechst 33258, Lucifer yellow, or quinine), radioactive labels (e.g., ³²P), electron-dense reagents, enzymes (e.g., peroxidase or alkaline phosphatase), biotin, fluorescent proteins (e.g., green fluorescent proteins), or haptens and proteins for which antisera or monoclonal antibodies are available. Labeling techniques are well known in the art.

### SNP Detection Kits

Certain embodiments are directed to kits that can be used to detect SNPs indicating the presence of one or more gene variants selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7) in a DNA sample. The disclosed kits include a binding molecule, such as an oligonucleotide probe that selectively hybridizes to the gene variant selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

Detection reagents can be developed and used to assay SNPs of the present invention (individually or in combination), and such detection reagents can be readily incorporated into a kit format. Accordingly, the present invention further provides SNP detection kits, including but not limited to, packaged probe and primer sets (e.g., TaqMan probe and primer sets), arrays and/or microarrays of nucleic acid molecules, and beads that contain one or more probes, primers, or other detection reagents for detecting one or more SNPs of the present invention. The kits can optionally include various electronic hardware components, containers, and devices.

The kit can further include one or more of a buffer solution, a conjugating solution for developing the signal of interest, or a detection reagent for detecting the signal of interest, each in separate packaging, such as a container. In another example, the kit includes a plurality of size-associated marker target nucleic acid sequences for hybridization with a detection array. The target nucleic acid sequences can include oligonucleotides such as DNA, RNA, and peptide-nucleic acid, or can include PCR fragments. The kit can also include instructions in a tangible form, such as written instructions or in a computer-readable format.

It will be readily apparent to one skilled in the art that the exact formulation of probes on an array is not critical as long as the user is able to select probes for inclusion on the array that fulfill the function of hybridizing to the targeted SNPs. The array can be modified to suit the needs of the user. Thus, analysis of the array can provide the user with information regarding the number and/or presence of protective alleles in a given sample. The hybridization of a probe complementary to a protective allele in an array can indicate that the subject from whom the sample was derived is at an elevated risk for developing a disease such as IgAN; or alternatively if it hybridizes to a protective allele the subject has a reduced risk.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mm (0.001 inch) to about 20 mm although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al. , Anal. Biochem. 217:306-10, 1994. In one example, the oligonucleotides are synthesized onto the support using conventional chemical techniques for preparing oligonucleotides on solid supports (see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90.degree. to permit synthesis to proceed within a second (2degrees) set of rows that are now perpendicular to the first set. This process creates a multiple-channel array whose intersection generates a plurality of discrete cells. In particular examples, the oligonucleotide probes on the array include one or more labels, which permit detection of oligonucleotide probe: target sequence hybridization complexes.

### REFERENCES

1. Task Force for Diagnosis and Treatment of Pulmonary Hypertension of European Society of Cardiology (ESC), European Respiratory Society (ERS), International Society of Heart and Lung Transplantation (ISHLT), et al. Guidelines for the diagnosis and treatment of pulmonary hypertension. Eur Respir J. 2009; 34:1219-63.
2. D′Alonzo G E, Barst R J, Ayres S M, et al. Survival in patients with primary pulmonary hypertension. Ann Int Med 1991; 115:343-9.
3. Girerd B, Montani D, Coulet F, et al. Clinical outcomes of pulmonary arterial hypertension in patients carrying an ACVRL1 (ALK1) mutation. Am J Respir Crit Care Med 2010; 181:851-61.
4. Deng Z, Morse J H, Slager S L, et al. Familial primary pulmonary hypertension (gene PPH1) is caused by mutations in the bone morphogenetic protein receptor-II gene. Am J Hum Genet 2000; 67:737-44.
5. Machado R D, Eickelberg O, Elliott C G, et al. Genetics and genomics of pulmonary arterial hypertension. J Am Coll Cardiol. 2009; 54(1 Suppl):532-42.
6. McAllister K A, Grogg K M, Johnson D W, et al. Endoglin, a TGF-beta binding protein of endothelial cells, is the gene for hereditary haemorrhagic telangiectasia type 1. Nat Genet 1994; 8:345-51.
7. Johnson D W, Berg J N, Baldwin M A, et al. Mutations in the activin receptor-like kinase 1 gene in hereditary haemorrhagic telangiectasia type 2. Nat Genet 1996; 13:189-95.
8. Abdalla S A, Pece-Barbara N, Vera S, et al. Analysis of ALK-1 and endoglin in newborns from families with hereditary hemorrhagic telangiectasia type 2. Hum Mol Genet 2000; 9:1227-37.
9. Harrison R E, Flanagan J A, Sankelo M, et al. Molecular and functional analysis identifies ALK-1 as the predominant cause of pulmonary hypertension related to hereditary haemorrhagic telangiectasia. J Med Genet 2003; 40:865-71.
10. Shintani M, Yagi H, Nakayama T, Saji T, Matsuoka R. A new nonsense mutation of SMAD8 associated with pulmonary arterial hypertension. J Med Genet 2009; 46:331-7.
11. Nasim M T, Ogo T, Ahmed M, et al. Molecular genetic characterization of SMAD signaling molecules in pulmonary arterial hypertension. Hum Mutat 2011; 32:1385-9.
12. Austin E D, Ma L, LeDuc C, et al. Whole exome sequencing to identify a novel gene (Caveolin-1) associated with human pulmonary arterial hypertension. Circ Cardiovasc Genet 2012; 5:336-43.
13. Li H and Durbin R. Fast and accurate short read alignment with Burrows-Wheeler transform. Bioinformatics 2009; 25: 1754-60.
14. Olschewski A, Li Y, Tang B, et al. Impact of KCNK3 in human pulmonary artery smooth muscle cells. Circ Res. 2006; 98:1072-80.
15. Hartness M E, Lewis A, Searle G J, O′Kelly I, Peers C, Kemp P J. Combined antisense and pharmacological approaches implicate hTASK as an airway O(2) sensing K(+) channel. J Biol Chem 2001; 276:26499-508.
16. Osipenko O N, Evans A M, Gurney A M. Regulation of the resting potential of rabbit pulmonary artery myocytes by a low threshold, O2-sensing potassium current. Br J Pharmacol 1997; 120:1461-70.
17. Czirjak G, Enyedi P. Formation of functional heterodimers between the TASK-1 and TASK-3 two-pore domain potassium channel subunits. J Biol Chem 2002; 277:5426-5432.
18. Patel A J, Honore E, Lesage F, Fink M, Romey G, Lazdunski M. Inhalational anesthetics activate two-pore-domain background K+ channels. Nat Neurosci 1999; 2:422-6.
19. U.S. Pat. No. 8,097,650.
20. Reyes R, Duprat F, Lesage F, et al. Cloning and expression of a novel pH-sensitive two pore domain K+ channel from human kidney. J Biol Chem 1998; 273:30863-9.
21. Gurney A M, Osipenko O N, MacMillan D, McFarlane K M, Tate R J, Kempsill F E. Two-pore domain K channel, TASK-1, in pulmonary artery smooth muscle cells. Circ Res 2003; 93:957-64.
22. Yu S P, Choi D W. Ions, cell volume, and apoptosis. Proc Natl Acad Sci USA 2000; 97:9360-9362.
23. Trapp S, Aller M I, Wisden W, Gourine A V. A role for TASK-1 (KCNK3) channels in the chemosensory control of breathing. J Neurosci 2008; 28:8844-50.
24. Gardener M J, Johnson I T, Burnham M P, Edwards G, Heagerty A M, Weston A H. Functional evidence of a role for two-pore domain potassium channels in rat mesenteric and pulmonary arteries. Br J Pharmacol 2004; 142:192-202.
25. Zuzarte M, Heusser K, Renigunta V, et al. Intracellular traffic of the K+ channels TASK-1 and TASK-3: role of N- and C-terminal sorting signals and interaction with 14-3-3 proteins. J Physiol 2009; 587:929-52.
26. Yuill K H, Stansfeld P J, Ashmole I, Sutcliffe M J, Stanfield P R. The selectivity, voltage-dependence and acid sensitivity of the tandem pore potassium channel TASK-1: contributions of the pore domains. Pflugers Arch. 2007; 455:333-48.
27. Streit A K, Netter M F, Kempf F, et al. A specific two-pore domain potassium channel blocker defines the structure of the TASK-1 open pore. J Biol Chem 2011; 286(16): 13977-84.
28. Yuan X J, Wang J, Juhaszova M, Gaine S P, Rubin L J. Attenuated K channel gene transcription in primary pulmonary hypertension. The Lancet, 1998; 9104:726-727.
29. Medhurst A D, Rennie G, Chapman C G, Meadows H, Duckworth M D, Kelsell R E, Gloger II, Pangalos M N. Distribution analysis of human two pore domain potassium channels in tissues of the central nervous system and periphery. Brain Res Mol Brain Res. 2001 Jan 31; 86(1-2):101-14.
30. Gaborit N, Le Bouter S, Szuts V, Varro A, Escande D, Nattel S, Demolombe S. Regional and tissue specific transcript signatures of ion channel genes in the non-diseased human heart. J Physiol. 2007 Jul 15; 582(Pt 2):675-93.
31. Moncada S, Gryglewsli R, Bunting S, Vane J R. An enzyme isolated from arteries transforms prostaglandin endoperoxides to an unstable substance that inhibits platelet aggregation. Nature 1976; 263:663-5.
32. Higenbottam T, Wheeldon D, Wells F, Wallwork J. Long-term treatment of primary pulmonary hypertension with continuous intravenous epoprostenol (prostacyclin). Lancet 1984; 1:1046-7.
33. Tang B, Li Y, Nagaraj C, et al. Endothelin-1 inhibits background two-pore domain channel TASK-1 in primary human pulmonary artery smooth muscle cells. Am J Respir Cell Mol Biol 2009; 41:476-83
34. Seyler C, Duthil-Straub E, Zitron E, et al. TASK1 (K(2P)3.1) K(+) channel inhibition by endothelin-1 is mediated through Rho kinase-dependent phosphorylation. Br J Pharmacol. 2012; 165(5):1467-75
35. Humbert M, Sitbon O, Simonneau G. Treatment of pulmonary arterial hypertension. N Engl J Med 2004; 351:1425-36.
36. O′Callaghan D S, Savale L, Montani D, et al. Treatment of pulmonary arterial hypertension with targeted therapies. Nat Rev Cardiol 2011; 8:526-38.
37. Atkinson C, Stewart S, Upton P D, et al. Primary pulmonary hypertension is associated with reduced pulmonary vascular expression of type II bone morphogenetic protein receptor. Circulation. 2002 Apr. 9; 105(14):1672-8.
38. Remillard C V, Tigno D D, Platoshyn O, et al. Function of Kv1.5 channels and genetic variations of KCNA5 in patients with idiopathic pulmonary arterial hypertension. Am J Physiol Cell Physiol 2007; 292:C1837-53.
39. Morecroft I, Murray A, Nilsen M, Gurney A M, MacLean M R. Treatment with the Kv7 potassium channel activator flupirtine is beneficial in two independent mouse models of pulmonary hypertension. Br J Pharmacol. 2009 August; 157(7):1241-9.
40. Li H and Durbin R. Fast and accurate short read alignment with Burrows-Wheeler transform. Bioinformatics 2009; 25:1754-60.
41. DePristo M A, Banks E, Poplin R et al, A framework for variation discovery and genotyping using next-generation DNA sequencing data. Nature Genetics 2011; 43:491-8
42. Bankston J R, Ye M, Chung W, et al. A novel and lethal de novo LQT-3 mutation in a newborn with distinct molecular pharmacology and therapeutic response. Plos One 2007; 2: e1258.
43. Miller A N, Long S B. Crystal structure of the human two-pore domain potassium channel K2P1. Science; 335:432-436
44. Nicholas K B, Nicholas H B Jr, and Deerfield D W II. GeneDoc: Analysis and Visualization of Genetic Variation, EMBNEW.NEWS 1997; 4:14.
45. Duprat F, Lesage F, Fink M, Reyes R, Heurteaux C, Lazdunski M. TASK, a human background K+ channel to sense external pH variations near physiological pH. Embo J 1997; 16:5464-5471.

What is claimed is:

1. A cDNA encoding one or more variants of a gene encoding KCNK3 having NM 002246 (SEQ. ID. NO. 1) selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

2. A method comprising
a) obtaining a biological sample from a subject,
b) determining if the biological sample
(i) comprises a loss-of-function variant of the Human KCNK3 channel gene having Gene Accession Number NM 002246, or
(ii) has significantly reduced KCNK3 function compared to a biological sample from a normal subject, or
(iii) expresses an mRNA encoding an abnormal form of KCNK3 protein, or
(iv) expresses a functionally abnormal KCNK3 channel protein,
c) determining that the subject is at risk for developing PAH if (i), (ii), (iii) or (iv) are satisfied in step b), and
d) if it is determined that the subject is at risk for developing PAH, then monitoring the subject for signs of PAH or treating the subject for PAH or both.

3. The method of claim 1, wherein treatment for PAH comprises prophylactic administration of a therapeutically effective amount of a KCNK3 channel agonist.

4. The method of claim 3, wherein the therapeutically effective amount of a KCNK3 agonist ranges from about 0.0001 to 100 mg/kg/day.

5. The method of claim 3, wherein the KCNK3 channel agonist is a member selected from the group consisting of ONO-RS-82, Treprostinil, Isoflurane and Halothane.

6. The method of claim 1, wherein the subject is asymptomatic.

7. The method of claim 3, wherein the subject is symptomatic.

8. The method of claim 7, wherein the symptomatic subject has increased pulmonary arterial pressure.

9. The method of claim 1, wherein the symptom of PAH is increased pulmonary arterial pressure.

10. The method of claim 3, wherein the subject has a familial history of PAH.

11. A method comprising
a) identifying a subject as having one or more symptoms of PAH,
b) obtaining a biological sample from the subject,
c) determining if the biological sample
(i) comprises a loss-of-function variant of the Human KCNK3 channel gene having Gene Accession Number NM 002246, or
(ii) has significantly reduced KCNK3 function compared to a biological sample from a normal subject, or
(iii) expresses an mRNA encoding an abnormal form of KCNK3 protein, or
(iv) expresses a functionally abnormal KCNK3 channel protein,
d) if it is determined that the biological sample from the subject satisfies (i), (ii), (iii),or
(iv), and the subject has increased pulmonary arterial pressure, then
f) diagnosing the subject as having PAH and treating the subject for PAH.

12. The method of claim 11, wherein the gene variant is selected from the group consisting of G203D, G97R, V221L, T8K, E182K, and Y192C.

13. The method of claim 11, wherein treating the subject for PAH comprises administering to the subject a therapeutically effective amount of a KCNK3 channel agonist.

14. The method of claim 11, wherein the KCNK3 channel agonist is a member selected from the group consisting of ONO-RS-82, treprostinil, isoflurane and halothane.

15. The method of claim 11, wherein the abnormal form of KCNK3 protein is a protein that has a characteristic selected from the group consisting of reduced potassium channel current at physiological pH, abnormal function or significantly reduced K+ channel current, abnormal topology based on the crystal structure of TWIK-1 channel, or reduced K⁺ selectivity.

16. The method of claim 11, wherein the subject has a familial history of PAH.

17. A method comprising
a) identifying a subject diagnosed with a disease or condition associated with significantly reduced KCNK3 function compared to a normal subject, and
b) administering to the subject a therapeutically effective amount of a KCNK3 channel agonist thereby treating the subject for the disease or condition.

18. Cells transfected with a cDNA encoding a loss-of-function variant selected from the group consisting of G203D, G97R, V221L, T8K, E182K, and Y192C.

19. A method comprising
a) identifying a subject having one or more symptoms of PAH,
b) obtaining a biological sample from the subject,
c) determining if the biological sample has significantly reduced KCNK3 function compared to a biological sample from a normal subject,
d) if it is determined that the biological sample from the subject satisfies c), then diagnosing the subject as at risk for developing PAH and monitoring the subject for symptoms of PAH or treating the subject for PAH.

20. The method of claim 19, wherein the treatment for PAH is administering the subject a therapeutically effective amount of a KCNK3 channel agonist that increases or normalizes KCNK3 function and increases pulmonary vasodilation.

21. The method of claim 19, wherein the loss-of-function variant is selected from the group consisting of G203D, G97R, V221L, T8K, E182K, and Y192C.

22. The method of claim 19, wherein the KCNK3 channel agonist is a member selected from the group consisting of ONO-RS-82, Treprostinil, Isoflurane and Halothane.

23. The method of claim 19, wherein the subject has a familial history of PAH.

24. A pharmaceutical formulation comprising two or more KCNK3 channel agonists, in therapeutically effective amounts that increase KCNK3 channel current in a pulmonary artery smooth muscle cell.

25. A kit comprising the pharmaceutical formulation of claim 24.

26. The pharmaceutical formulation of claim 25, wherein the KCNK3 channel agonist is a member selected from the group consisting of ONO-RS-82, Treprostinil, Isoflurane and Halothane.

27. A microarray comprising one or more oligonucleotide probes bound to a support which probes are complementary to and hybridize to one or more respective target oligonucleotide capable of selectively hybridizing to one or more nucleic acid molecules having a single nucleotide polymorphism (SNP) in the KCNK3 gene having NM 002246 (SEQ. ID. NO. 1) wherein said SNP is selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

28. A kit comprising an assay for detecting at least one SNP in the gene encoding KCNK3 having NM 002246 (SEQ. ID. NO. 1) in a biological sample obtained from a subject, wherein said SNP is selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7) and the presence of at least one SNP identifies said subject as having an increased risk for developing PAH.

29. The kit of claim 28 wherein said assay comprises nucleic acid probes and/or primers specific to said at least one SNP selected from the group consisting of E182K (SEQ. ID. NO. 2), T8K (SEQ. ID. NO. 3), Y192C (SEQ. ID. NO. 4), G203D (SEQ. ID. NO. 5), G97R (SEQ. ID. NO. 6), and V221L (SEQ. ID. NO. 7).

30. The kit of claim 28, further comprising instructions for correlating said assay results with said subject's risk for having or developing PAH.

31. The method of claim 2, wherein detecting the loss-of-function variant of the Human KCNK3 channel gene in the biological sample is accomplished using one or more of oligonucleotide microarray analysis, allele-specific hybridization, allele-specific polymerase chain reaction (PCR), 5′ nuclease digestion, molecular beacon assay, oligonucleotide ligation assay, size analysis, or nucleic acid sequencing.

32. The microarray of claim 27, wherein the oligonucleotide hybridization occurs under stringent conditions.

33. A method comprising
a) identifying a subject diagnosed with PAH, and
b) administering to the subject a therapeutically effective amount of a KCNK3 channel agonist thereby treating the subject for the disease or condition.


SEQUENCE LISTING (7 sequences)

SEQ ID NO: 1 - DNA, 1185 bp, Homo sapiens
  misc_feature: Gene encoding KCNK3
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagcctgtccaccttccgcggcctcatgaagcgcaggagctccgtgtga

SEQ ID NO: 2 - DNA, 1140 bp, Homo sapiens
  misc_feature: KCNK3 variant E182K
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactgctgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc

SEQ ID NO: 3 - DNA, 1140 bp, Homo sapiens
  misc_feature: KCNK3 variant T8K
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacaggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc

SEQ ID NO: 4 - DNA, 1140 bp, Homo sapiens
  misc_feature: KCNK3 variant Y192C
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacctggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc

SEQ ID NO: 5 - DNA, 1140 bp, Homo sapiens
  misc_feature: KCNK3 variant G203D
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcgacgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc

SEQ ID NO: 6 - DNA, 1140 bp, Homo sapiens
  misc_feature: KCNK3 variant G97R
atgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacaagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc

SEQ ID NO: 7 - DNA, 1140 bp, Homo sapiens
  misc_feature: KCNK3 variant V221L
atgaagcggcagaacgtgcgcaagctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagc